(19) United States
(12) Patent Application Publication
(10) Pub. No.: US 2022/0117513 A1
(43) Pub. Date: Apr. 21, 2022

(54) MICROBIOME-DERIVED GASEOUS SAMPLE COLLECTION SYSTEM AND METHODS

(71) Applicants: Cedars-Sinai Medical Center, Los Angeles, CA (US); Gemelli Biotech Corp., Raleigh, NC (US)

(72) Inventors: Aniruddh PANDIT, Los Angeles, CA (US); Mark PIMENTEL, Los Angeles, CA (US); Kapil GUPTA, Santa Monica, CA (US); Matthew MITCHO, Raleigh, NC (US); Ali REZAIE, Beverly Hills, CA (US)

(21) Appl. No.: 17/503,041

(22) Filed: Oct. 15, 2021

Related U.S. Application Data
(60) Provisional application No. 63/093,088, filed on Oct. 16, 2020.

(51) Int. Cl. A61B 5/097; A61B 5/08; A61B 10/00; B01L 3/00

(52) U.S. Cl. CPC A61B 5/097; A61B 5/082; A61B 10/0096; B01L 2300/12; A61B 2010/0087; B01L 2300/049; B01L 2300/0848; B01L 3/505

(57) ABSTRACT

Provided are systems and methods for collecting and storing a microbiome-derived gaseous sample.

### CROSS-REFERENCE

This application claims the benefit of U.S. Provisional Patent Application No. 63/093,088 filed on Oct. 16, 2020, which is incorporated by reference in its entirety.

### SUMMARY

Breath testing to detect gases produced by the gut microbiome can be used to determine diseases or disorders such as small intestinal bacterial overgrowth (SIBO) or diarrhea. Levels of gases such as hydrogen (H₂), methane (CH₄), and hydrogen sulfide (H₂S) may be used to make these determinations. However, conventional breath tests do not measure all these gases or suffer from accuracy and sensitivity. Thus, there is a need for improved gas sample collection systems.

Described herein, in some embodiments, are systems, methods, and devices for measuring hydrogen, methane, and hydrogen sulfide for making an accurate determination of a disease or disorder (e.g., SIBO, diarrhea). Systems, methods, and devices, described herein, may comprise improved collection bags for collecting the gases and storing the gases for longer periods of time.

An aspect described herein are devices collecting a microbiome-derived gaseous sample, comprising: a multilayer composite forming a bag having an interior space for containing the microbiome-derived gaseous sample, the multilayer composite comprising: an inner layer comprising a first polymer film, a second layer, adjacent to the inner layer, comprising a foil, a third layer, adjacent to the second layer, comprising a second polymer film, and an exterior layer, adjacent to the third layer; and a valve for in fluid connection with the interior space of the bag, wherein the valve is a one-way valve. In one feature, the first polymer film comprises low-density polyethylene. In one feature, the first polymer film comprises linear low-density polyethylene. In one feature, the foil is an aluminum foil. In one feature, the inner layer and the second layer comprise a metalized film. In one feature, the metalized film comprises biaxially-oriented polyethylene terephthalate. In one feature, the foil is an aluminum foil. In one feature, the third layer and the second layer comprise a metalized film. In one feature, the metalized film comprises biaxially-oriented polyethylene terephthalate. In one feature, the exterior layer comprises nylon. In one feature, the exterior layer comprises a biaxially oriented nylon film (BON). In one feature, the devices further comprise a mouthpiece. In one feature, the exterior layer is approximately 0.0006 inches thick. In one feature, the interior layer is approximately 0.00225 inches thick. In one feature, the second layer is approximately 0.0003 inches thick. In one feature, the third layer is approximately 0.00015 inches thick. In one feature, the bag is a flat bag. In one feature, the bag is formed by two layers of the multilayer composite, and wherein the interior space is formed by a seal between the two layers. In one feature, the seal has a width of approximately 0.375 inches. In one feature, the seal is formed by heat sealing. In one feature, the heat sealing comprises applying a temperature of approximately 400 degrees Fahrenheit and a pressure of approximately 40 pounds per square inch for a dwell time of approximately 1.5 seconds to form the seal. In one feature, the interior space comprises a width of approximately 4 inches, and a length of approximately 6 inches. In one feature, an aperture having a diameter of approximately 0.375 inches is formed through one layer of the multilayer composite to receive the valve. In one feature, the aperture is located approximately 1.25 inches from a side of the seal which forms the width of the interior space, and wherein the aperture is located approximately 2 inches from a side of the seal which forms the length of the interior space. In one feature, the device comprises a seal strength of approximately 18 pounds per inch. In one feature, the device comprises a tensile strength of approximately 19 pounds per inch. In one feature, the device comprises a puncture resistance of approximately 20 pounds. In one feature, the device comprises an oxygen transmission rate of approximately 0.0005 cubic centimeters per 100 square inches per day at 0% relative humidity and 23 degrees Celsius. In one feature, the device comprises a water vapor transmission rate of approximately 0.0005 grams per 100 square inches per day at 90% relative humidity and 40 degrees Celsius. In one feature, the microbiome-derived gaseous sample is a breath sample. In one feature, the microbiome-derived gaseous sample comprises one or more gases. In one feature, the one or more gases comprises carbon dioxide, hydrogen, methane, hydrogen sulfide, or combinations thereof. In one feature, a first gas of the one or more gases is hydrogen sulfide. In one feature, the one or more gases comprises carbon dioxide and hydrogen sulfide. In one feature, the one or more gases comprises hydrogen and hydrogen sulfide. In one feature, the one or more gases comprises methane and hydrogen sulfide. In one feature, the one or more gases comprises hydrogen, methane, and hydrogen sulfide. In one feature, the device retains at least or about 80% of the carbon dioxide, hydrogen, methane, hydrogen sulfide, or combinations thereof after at least 24 hours. In one feature, the device retains at least or about 80% of the carbon dioxide, hydrogen, methane, hydrogen sulfide, or combinations thereof after at least 48 hours. In one feature, the device retains at least or about 80% of the carbon dioxide, hydrogen, methane, hydrogen sulfide, or combinations thereof after at least 1 week. In one feature, the device retains at least or about 80% of the hydrogen, methane, and hydrogen sulfide after at least 24 hours. In one feature, the device retains at least or about 80% of the hydrogen, methane, and hydrogen sulfide after at least 48 hours. In one feature, the device retains at least or about 80% of the hydrogen, methane, and hydrogen sulfide after at least 1 week.

An aspect described herein are methods for assessing a microbiome of an individual, the method comprising: a) collecting a sample from the individual using a device of described herein; b) detecting one or more gases in the sample; and c) assessing the microbiome of the individual based on the detection of the one or more gases in the sample. In one feature, the one or more gases comprises carbon dioxide, hydrogen, methane, or hydrogen sulfide. In one feature, a first gas of the one or more gases is hydrogen sulfide. In one feature, the one or more gases comprises carbon dioxide and hydrogen sulfide. In one feature, the one or more gases comprises hydrogen and hydrogen sulfide. In one feature, the one or more gases comprises methane and hydrogen sulfide. In one feature, the one or more gases comprises hydrogen, methane, and hydrogen sulfide. In one feature, the method further comprises following step c), determining whether an individual has a disease or disorder based on the detection of the one or more gases in the sample. In one feature, the disease or disorder is a metabolic disorder. In one feature, the disease or disorder is diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, glomerular disease, or intestinal methanogenic overgrowth. In one feature, if the one or more gases are elevated by at least about 2-fold, then the individual is determined to have the disease or disorder.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 depicts a collection device of a microbiome-derived gaseous sample collection system, according to some embodiments;

FIG. 2 depicts a composite material of a collection device, according to some embodiments;

FIG. 3A depicts a mouthpiece component of a microbiome-derived gaseous sample collection system, according to some embodiments;

FIG. 3B depicts a mouthpiece component of a microbiome-derived gaseous sample collection system, according to some embodiments;

FIGS. 4A-4C show graphs depicting the stability of various gaseous of a microbiome-derived gaseous sample being retained within a sample collection bag, according to some embodiments herein from an Experiment 1 (FIG. 4A), an Experiment 2 (FIG. 4B), and an Experiment 3 (FIG. 4C);

FIG. 5 shows data obtained from a stability test of a preexisting bag used to collect gaseous samples;

FIG. 6A shows a comparison of maximum exhaled H₂S levels among the three groups. H₂S level ≥5 parts per million (ppm) at any point of the test (dashed line) differentiates diarrhea from constipation patients with sensitivity and specificity of 75% and 94.8%, respectively; and

FIG. 6B shows H₂S AUC levels during breath testing of healthy subjects, constipation and diarrhea groups.

### DETAILED DESCRIPTION

Provided herein are embodiments of a sample collection system for collecting gaseous samples. In some embodiments, the gaseous samples are utilized to evaluate the microbiome of a subject or patient. The gaseous samples may be collected using the systems and methods described herein. In some embodiments, the collection system is utilized to evaluate levels of hydrogen, methane, and hydrogen sulfide contained in the gaseous sample. In some embodiments, levels of carbon dioxide are evaluated to standardize the levels of other gases collected by the system.

### Collection Bag

In some embodiments, provided herein is a sample collection device. In some embodiments, the device comprises a collection bag to capture a gaseous sample from a patient. In some embodiments, a patient breathes or blows into the collection bag to supply the gaseous sample into the bag. The collection bag may also be referred to as a breath bag.

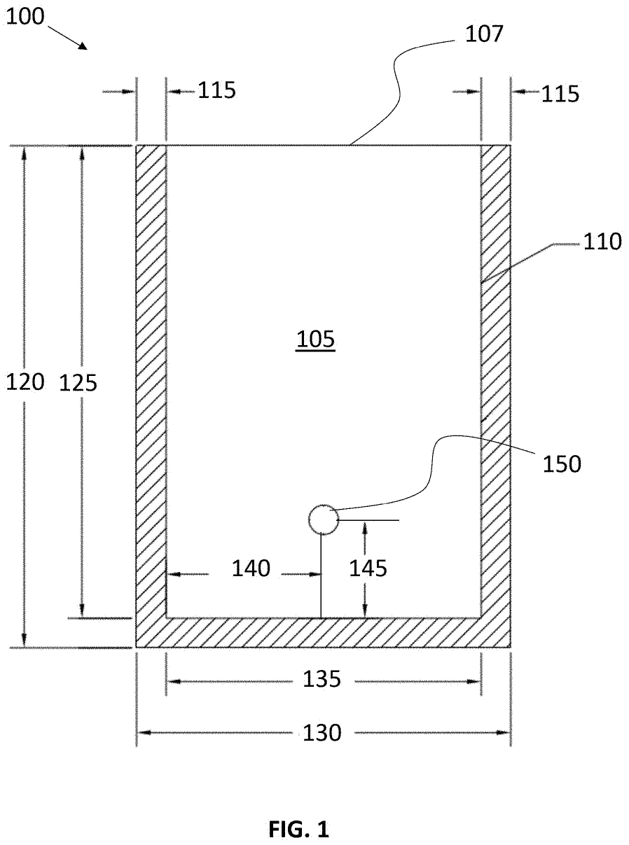

With reference to FIG. 1, a collection device 100 is depicted, according to some embodiments. In some embodiments, the collection bag 100 comprises an interior space 105 formed by a seal 110. In some embodiments, the bag 100 is provided to a subject in a substantially flat form and the interior space 105 inflates as the gaseous sample is collected from the subject.

In some embodiments, the bag 100 is formed by a unique multilayer composite material as described herein. In some embodiments, the bag 100 is formed by a single sheet of composite folded onto itself and sealed. In said embodiments, three sides of the interior space 105 are formed by the seal 110 and one side of the interior space is formed by a fold 107 in the composite material. In some embodiments, the bag is formed by two sheets of composite material and all four sides of the interior space are formed by a seal. In some embodiments, excess material is trimmed from the outer edge of the seal 110 to form the bag.

In some embodiments, the seal 110 is created by a heat seal. In some embodiments, a temperature of approximately 400 degrees Fahrenheit (° F.) at a pressure of approximately 40 pounds per square inch (psi) is applied for 1.5 seconds to form the seal 110. It may be acknowledged that different conditions may suffice to form seal 110 with to meet the requirements of the sample collection devices provided herein.

In some embodiments, a seal thickness 115 is sufficient to meet the requirements of the collection system. In some embodiments, the exterior dimensions of a collection bag 100 comprise an exterior length 120 and an exterior width 130. In some embodiments, the collection bag is substantially rectangular and the width of the bag is less than the length of the bag. In some embodiments, the collection bag is substantially square and the width of the bag is approximately equal to the length of the bag. The collection bag may comprise any shape suitable for collecting a gaseous sample using methods as described herein.

In some embodiments, the thickness of the seal 115 is approximately 0.375 inches. In some embodiments, the thickness of the seal is about 0.125 inches to about 1 inch. In some embodiments, the thickness of the seal is about 0.125 inches to about 0.25 inches, about 0.125 inches to about 0.375 inches, about 0.125 inches to about 0.5 inches, about 0.125 inches to about 0.625 inches, about 0.125 inches to about 0.875 inches, about 0.125 inches to about 1 inch, about 0.25 inches to about 0.375 inches, about 0.25 inches to about 0.5 inches, about 0.25 inches to about 0.625 inches, about 0.25 inches to about 0.875 inches, about 0.25 inches to about 1 inch, about 0.375 inches to about 0.5 inches, about 0.375 inches to about 0.625 inches, about 0.375 inches to about 0.875 inches, about 0.375 inches to about 1 inch, about 0.5 inches to about 0.625 inches, about 0.5 inches to about 0.875 inches, about 0.5 inches to about 1 inch, about 0.625 inches to about 0.875 inches, about 0.625 inches to about 1 inch, or about 0.875 inches to about 1 inch. In some embodiments, the thickness of the seal is about 0.125 inches, about 0.25 inches, about 0.375 inches, about 0.5 inches, about 0.625 inches, about 0.875 inches, or about 1 inch. In some embodiments, the thickness of the seal is at least about 0.125 inches, about 0.25 inches, about 0.375 inches, about 0.5 inches, about 0.625 inches, or about 0.875 inches. In some embodiments, the thickness of the seal is at most about 0.25 inches, about 0.375 inches, about 0.5 inches, about 0.625 inches, about 0.875 inches, or about 1 inch.

In some embodiments, the exterior length 120 comprises a distance from the folded side 107 of the bag to the outer edge of seal 110 opposite of the folded edge. In some embodiments, the exterior length comprises a distance from the outer edge of the seal to the opposing outer edge of the seal (e.g. in embodiments wherein two sheets of material are used to form the bag and a seal is created on four sides to form the interior space). In some embodiments, the exterior width 130 of the bag 100 comprises a distance from the outer edge of the seal to the opposing outer edge of the seal along the width of the bag 100.

In some embodiments, the exterior length 120 of the bag 100 is approximately 6.375 inches. In some embodiments, the exterior length 120 of the bag 100 is about 2 inches to about 10 inches. In some embodiments, the exterior length 120 of the bag 100 is about 1 inches to about 20 inches, about 2 inches to about 20 inches, about 3 inches to about 20 inches, about 4 inches to about 20 inches, about 5 inches to about 20 inches, about 6 inches to about 20 inches, about 7 inches to about 20 inches, about 8 inches to about 20 inches, about 9 inches to about 20 inches, about 10 inches to about 20 inches, about 12 inches to about 20 inches, about 14 inches to about 20 inches, about 16 inches to about 20 inches, about 18 inches to about 20 inches, about 1 inch to about 10 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 1 inch to about 8 inches, about 2 inches to about 8 inches, about 3 inches to about 8 inches, about 4 inches to about 8 inches, about 5 inches to about 8 inches, or about 6 inches to about 8 inches. In some embodiments, the exterior length 120 of the bag 100 is about or at least 1 inch, 2 inches, 3 inches, 4 inches, 5 inches, 6 inches, 7 inches, 8 inches, 9 inches, 10 inches, 12 inches, 13 inches, 14 inches, or more.

In some embodiments, the exterior width 130 of the bag 100 is approximately 4.750 inches. In some embodiments, the exterior width 130 of the bag 100 is approximately 6.375 inches. In some embodiments, the exterior width 130 of the bag 100 is about 2 inches to about 10 inches. In some embodiments, the exterior width 130 of the bag 100 is about 1 inches to about 20 inches, about 2 inches to about 20 inches, about 3 inches to about 20 inches, about 4 inches to about 20 inches, about 5 inches to about 20 inches, about 6 inches to about 20 inches, about 7 inches to about 20 inches, about 8 inches to about 20 inches, about 9 inches to about 20 inches, about 10 inches to about 20 inches, about 12 inches to about 20 inches, about 14 inches to about 20 inches, about 16 inches to about 20 inches, about 18 inches to about 20 inches, about 1 inch to about 10 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 1 inch to about 8 inches, about 2 inches to about 8 inches, about 3 inches to about 8 inches, about 4 inches to about 8 inches, about 5 inches to about 8 inches, or about 6 inches to about 8 inches. In some embodiments, the exterior width 130 of the bag 100 is about or at least 1 inch, 2 inches, 3 inches, 4 inches, 5 inches, 6 inches, 7 inches, 8 inches, 9 inches, 10 inches, 12 inches, 13 inches, 14 inches, or more.

In some embodiments, the interior of the bag 105 is formed by an interior length 125 and an interior width of the bag 135. In some embodiments, the interior length 125 of the bag comprises a distance from the folded side 107 of the bag to the inner edge of seal 110 opposite of the folded edge. In some embodiments, the interior length comprises a distance from the inner edge of the seal to the opposing inner edge of the seal (e.g. in embodiments wherein two sheets of material are used to form the bag and a seal is created on four sides to form the interior space). In some embodiments, the interior width 135 comprises a distance from the inner edge of the seal 110 to the opposing inner edge of the seal 110.

In some embodiments, the interior length 125 of the bag 100 is approximately 6.000 inches. In some embodiments, the interior length 125 of the bag 100 is about 2 inches to about 10 inches. In some embodiments, the interior length 125 of the bag 100 is about 1 inches to about 20 inches, about 2 inches to about 20 inches, about 3 inches to about 20 inches, about 4 inches to about 20 inches, about 5 inches to about 20 inches, about 6 inches to about 20 inches, about 7 inches to about 20 inches, about 8 inches to about 20 inches, about 9 inches to about 20 inches, about 10 inches to about 20 inches, about 12 inches to about 20 inches, about 14 inches to about 20 inches, about 16 inches to about 20 inches, about 18 inches to about 20 inches, about 1 inch to about 10 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 1 inch to about 8 inches, about 2 inches to about 8 inches, about 3 inches to about 8 inches, about 4 inches to about 8 inches, about 5 inches to about 8 inches, or about 6 inches to about 8 inches. In some embodiments, the interior length 125 of the bag 100 is about or at least 1 inch, 2 inches, 3 inches, 4 inches, 5 inches, 6 inches, 7 inches, 8 inches, 9 inches, 10 inches, 12 inches, 13 inches, 14 inches, or more.

In some embodiments, the interior width of the bag 100 is approximately 4.000 inches. In some embodiments, the interior width of the bag 100 is about 2 inches to about 10 inches. In some embodiments, the interior width of the bag 100 is about 1 inches to about 20 inches, about 2 inches to about 20 inches, about 3 inches to about 20 inches, about 4 inches to about 20 inches, about 5 inches to about 20 inches, about 6 inches to about 20 inches, about 7 inches to about 20 inches, about 8 inches to about 20 inches, about 9 inches to about 20 inches, about 10 inches to about 20 inches, about 12 inches to about 20 inches, about 14 inches to about 20 inches, about 16 inches to about 20 inches, about 18 inches to about 20 inches, about 1 inch to about 10 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 1 inch to about 8 inches, about 2 inches to about 8 inches, about 3 inches to about 8 inches, about 4 inches to about 8 inches, about 5 inches to about 8 inches, or about 6 inches to about 8 inches. In some embodiments, the interior width of the bag 100 is about or at least 1 inch, 2 inches, 3 inches, 4 inches, 5 inches, 6 inches, 7 inches, 8 inches, 9 inches, 10 inches, 12 inches, 13 inches, 14 inches, or more.

In some embodiments, the bag 100 comprises a valve 150 in fluid connection with the interior 105 of the bag 100. In some embodiments, the valve 150 is a one-way valve, such that the gaseous sample flows from the subject into the interior space 105 and does not escape back out of the valve 150. In some embodiments, the valve 150 is configured to receive an end of a mouth piece, as described herein.

In some embodiments, valve 150 is positioned at the approximate center of the width of the bag. In some embodiments, the distance 140 from an interior edge of the seal 110 of the length of the bag to the center of the valve 150 is approximately two inches. In some embodiments, the distance 140 from an interior edge of the seal 110 of the length of the bag to the center of the valve 150 is about 1 inch to about 10 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 1 inch to about 8 inches, about 2 inches to about 8 inches, about 3 inches to about 8 inches, about 4 inches to about 8 inches, about 5 inches to about 8 inches, or about 6 inches to about 8 inches. In some embodiments, the distance 140 from an interior edge of the seal 110 of the length of the bag to the center of the valve 150 is about or at least 1 inch, 2 inches, 3 inches, 4 inches, 5 inches, 6 inches, 7 inches, 8 inches, 9 inches, 10 inches, 12 inches, 13 inches, 14 inches, or more. In some embodiments, the valve 150 is provided closer to the side of the bag opposing the folded side 107. In some embodiments, the distance 145 from the valve 150 to the side of the bag opposing the folded side is approximately 1.250 inches. In some embodiments, the distance 145 from the valve 150 to the side of the bag opposing the folded side is about 1 inch to about 10 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 1 inch to about 8 inches, about 2 inches to about 8 inches, about 3 inches to about 8 inches, about 4 inches to about 8 inches, about 5 inches to about 8 inches, or about 6 inches to about 8 inches. In some embodiments, the distance 145 from the valve 150 to the side of the bag opposing the folded side is about or at least 1 inch, 2 inches, 3 inches, 4 inches, 5 inches, 6 inches, 7 inches, 8 inches, 9 inches, 10 inches, 12 inches, 13 inches, 14 inches, or more.

In some embodiments, the diameter of valve is approximately 0.375 inches. In some embodiments, the diameter of valve is about 0.125 inches to about 1 inch. In some embodiments, the diameter of valve is about 0.125 inches to about 0.25 inches, about 0.125 inches to about 0.375 inches, about 0.125 inches to about 0.5 inches, about 0.125 inches to about 0.625 inches, about 0.125 inches to about 0.875 inches, about 0.125 inches to about 1 inch, about 0.25 inches to about 0.375 inches, about 0.25 inches to about 0.5 inches, about 0.25 inches to about 0.625 inches, about 0.25 inches to about 0.875 inches, about 0.25 inches to about 1 inch, about 0.375 inches to about 0.5 inches, about 0.375 inches to about 0.625 inches, about 0.375 inches to about 0.875 inches, about 0.375 inches to about 1 inch, about 0.5 inches to about 0.625 inches, about 0.5 inches to about 0.875 inches, about 0.5 inches to about 1 inch, about 0.625 inches to about 0.875 inches, about 0.625 inches to about 1 inch, or about 0.875 inches to about 1 inch. In some embodiments, the thickness of the seal is about 0.125 inches, about 0.25 inches, about 0.375 inches, about 0.5 inches, about 0.625 inches, about 0.875 inches, or about 1 inch. In some embodiments, the diameter of valve is at least about 0.125 inches, about 0.25 inches, about 0.375 inches, about 0.5 inches, about 0.625 inches, or about 0.875 inches. In some embodiments, the thickness of the seal is at most about 0.25 inches, about 0.375 inches, about 0.5 inches, about 0.625 inches, about 0.875 inches, or about 1 inch. In some embodiments, the valve 150 is configured to receive an end of a mouth piece, as described herein.

### Multilayer Composite Material

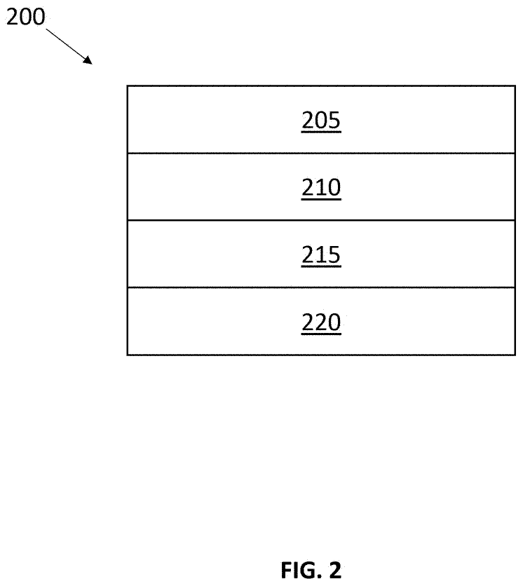

In some embodiments, the collection device is formed by a multilayer composite material. FIG. 2 depicts a cross-section of the multilayer composite material 200, according to some embodiments. In some embodiments, the multilayer composite material 200 is CADPAK-N or MIL-PRF-131K Class 1. In some embodiments, the composite material 200 comprises an inner layer 220 forming the interior space of the bag to be in contact with the gaseous sample. In some embodiments, the composite material 200 comprises an exterior layer 205 forming the exterior of the bag and to be in contact with the environment.

In some embodiments, the inner layer 220 is selected to be compatible with a gaseous sample. The inner layer material may be selected to be inert with components of the gaseous sample. In some embodiments, the inner layer material is selected to not disrupt, alter, or react with levels of the components of the gaseous sample. In some embodiments, the inner layer 220 comprises a material that will not react with or alter levels of carbon dioxide, hydrogen, methane, and hydrogen sulfide contained within a gaseous sample.

In some embodiments, the inner layer 220 comprises a polymer film. In some embodiments, the inner layer 220 comprises polyethylene (PE). In some embodiments, the inner layer 220 comprises low-density polyethylene (LDPE). In some embodiments, the interior layer is approximately 0.00225 inches thick or approximately 2.25 mil thick.

In some embodiments, the exterior layer material is selected to be puncture resistant. In some embodiments, the exterior layer is puncture resistant to at least approximately 20 pounds and in accordance with military standard 3010. In some embodiments, the exterior layer 205 comprises a nylon film. In some embodiments, the exterior layer comprises a biaxially oriented nylon film (BON). In some embodiments, the exterior layer 205 is approximately 0.0006 inches thick or having a thickness of about 60 gauge (ga).

In some embodiments, the composite material 200 further comprises a second layer 215. The second layer 215 may be adjacent to the inner layer. In some embodiments, the second layer 215 comprises a reflective characteristic to prevent light from entering into the interior space of the bag and reacting with a gaseous sample contained within. In some embodiments, the second layer provides 215 an element of thermal resistance to better maintain a temperature of a gaseous sample located within an interior space of the bag.

In some embodiments, the second layer 215 comprises a foil. In some embodiments, the second layer 215 comprises a metalized film. In some embodiments, the second layer 215 comprises an aluminum foil. In some embodiments, the second layer 215 comprises biaxially-oriented polyethylene terephthalate. In some embodiments, the second layer is approximately 0.0003 inches or 30 gauge (ga) thick.

In some embodiments, the composite material 200 further comprises a third layer 210. In some embodiments, the third layer is adjacent to the exterior layer 205. In some embodiments, the third layer 210 is provided between the second layer 215 and the exterior layer 205. In some embodiments, the third layer comprises a polymer film. In some embodiments, the third layer 205 comprises polyethylene (PE). In some embodiments, the third layer 205 comprises low-density polyethylene (LDPE). In some embodiments, the third layer is approximately 0.00015 inches thick or approximately 15 gauge (ga).

### Collection Bag

In some embodiments, the collection bag is configured to receive and store a gaseous sample obtained from a subject. In some embodiments, the bag is formed from a multilayer composite material. The inner layer of the composite material may be selected from a material which will inert to the gases which comprise the gaseous sample, such that the inner layer material will not affect levels of the gases of the gaseous sample when tested and analyzed.

In some embodiments, a seal which forms the inner layer of the bag is resistant to forces of at least 18 pounds per square inch (PSI). In some embodiments, a seal strength of at least 18 psi ensures that the seal of the bag will not break or rupture during handling and transportation of a collection bag containing a gaseous sample within. In some embodiments, the one way valve is also resistant to forces of at least 18 psi to ensure that the valve will not release gas contained within when such forces are experienced.

In some embodiments, seal strength of the collection device is about 15 psi to about 40 psi. In some embodiments, seal strength of the collection device is about 15 psi to about 18 psi, about 15 psi to about 20 psi, about 15 psi to about 25 psi, about 15 psi to about 30 psi, about 15 psi to about 35 psi, about 15 psi to about 40 psi, about 18 psi to about 20 psi, about 18 psi to about 25 psi, about 18 psi to about 30 psi, about 18 psi to about 35 psi, about 18 psi to about 40 psi, about 20 psi to about 25 psi, about 20 psi to about 30 psi, about 20 psi to about 35 psi, about 20 psi to about 40 psi, about 25 psi to about 30 psi, about 25 psi to about 35 psi, about 25 psi to about 40 psi, about 30 psi to about 35 psi, about 30 psi to about 40 psi, or about 35 psi to about 40 psi. In some embodiments, seal strength of the collection device is about 15 psi, about 18 psi, about 20 psi, about 25 psi, about 30 psi, about 35 psi, or about 40 psi. In some embodiments, seal strength of the collection device is at least about 15 psi, about 18 psi, about 20 psi, about 25 psi, about 30 psi, or about 35 psi.

In some embodiments, the seal and composite material which form the bag comprises a tensile strength of at least about 19 pounds per inch (ppi). In some embodiments, a tensile strength of at least about 19 ppi ensures that the collection bag will not tear, pull apart, or otherwise fail due to tensile forces experienced during handling and transportation of a collection bag containing a gaseous sample within.

In some embodiments, the tensile strength of the collection device is about 15 ppi to about 45 ppi. In some embodiments, the tensile strength of the collection device is about 15 ppi to about 20 ppi, about 15 ppi to about 25 ppi, about 15 ppi to about 30 ppi, about 15 ppi to about 35 ppi, about 15 ppi to about 40 ppi, about 15 ppi to about 45 ppi, about 20 ppi to about 25 ppi, about 20 ppi to about 30 ppi, about 20 ppi to about 35 ppi, about 20 ppi to about 40 ppi, about 20 ppi to about 45 ppi, about 25 ppi to about 30 ppi, about 25 ppi to about 35 ppi, about 25 ppi to about 40 ppi, about 25 ppi to about 45 ppi, about 30 ppi to about 35 ppi, about 30 ppi to about 40 ppi, about 30 ppi to about 45 ppi, about 35 ppi to about 40 ppi, about 35 ppi to about 45 ppi, or about 40 ppi to about 45 ppi. In some embodiments, the tensile strength of the collection device is about 15 ppi, about 20 ppi, about 25 ppi, about 30 ppi, about 35 ppi, about 40 ppi, or about 45 ppi. In some embodiments, the tensile strength of the collection device is at least about 15 ppi, about 20 ppi, about 25 ppi, about 30 ppi, about 35 ppi, or about 40 ppi.

In some embodiments, the collection device comprises a puncture resistance of at least 20 pounds (lbs). In some embodiments, a puncture resistance of at least about 20 lbs ensures that the collection bag will not be punctured during handling and transportation of a collection bag containing a gaseous sample within.

In some embodiments, the puncture resistance of the collection device is about 15 lbs to about 45 lbs. In some embodiments, the puncture resistance of the collection device is about 15 lbs to about 20 lbs, about 15 lbs to about 25 lbs, about 15 lbs to about 30 lbs, about 15 lbs to about 35 lbs, about 15 lbs to about 40 lbs, about 15 lbs to about 45 lbs, about 20 lbs to about 25 lbs, about 20 lbs to about 30 lbs, about 20 lbs to about 35 lbs, about 20 lbs to about 40 lbs, about 20 lbs to about 45 lbs, about 25 lbs to about 30 lbs, about 25 lbs to about 35 lbs, about 25 lbs to about 40 lbs, about 25 lbs to about 45 lbs, about 30 lbs to about 35 lbs, about 30 lbs to about 40 lbs, about 30 lbs to about 45 lbs, about 35 lbs to about 40 lbs, about 35 lbs to about 45 lbs, or about 40 lbs to about 45 lbs. In some embodiments, the puncture resistance of the collection device is about 15 lbs, about 20 lbs, about 25 lbs, about 30 lbs, about 35 lbs, about 40 lbs, or about 45 lbs. In some embodiments, the puncture resistance of the collection device is at least about 15 lbs, about 20 lbs, about 25 lbs, about 30 lbs, about 35 lbs, or about 40 lbs.

In some embodiments, the collection device comprises an oxygen transmission rate of approximately 0.0005 cubic centimeters (cc) or less per 100 square inches per day at 0% relative humidity and 23 degrees Celsius. In some embodiments, an oxygen transmission rate of 0.0005 cc or less ensures that the gaseous sample is not contaminated or that the gaseous sample is not reduced during handling or transportation of the collection bag.

In some embodiments, the collection device comprises an oxygen transmission rate of about 0.00001 cc to about 0.0005 cc. In some embodiments, the collection device comprises an oxygen transmission rate of about 0.0005 cc to about 0.0003 cc, about 0.0005 cc to about 0.0001 cc, about 0.0005 cc to about 0.00005 cc, about 0.0005 cc to about 0.00003 cc, about 0.0005 cc to about 0.00001 cc, about 0.0003 cc to about 0.0001 cc, about 0.0003 cc to about 0.00005 cc, about 0.0003 cc to about 0.00003 cc, about 0.0003 cc to about 0.00001 cc, about 0.0001 cc to about 0.00005 cc, about 0.0001 cc to about 0.00003 cc, about 0.0001 cc to about 0.00001 cc, about 0.00005 cc to about 0.00003 cc, about 0.00005 cc to about 0.00001 cc, or about 0.00003 cc to about 0.00001 cc. In some embodiments, the collection device comprises an oxygen transmission rate of about 0.0005 cc, about 0.0003 cc, about 0.0001 cc, about 0.00005 cc, about 0.00003 cc, or about 0.00001 cc. In some embodiments, the collection device comprises an oxygen transmission rate of at most about 0.0003 cc, about 0.0001 cc, about 0.00005 cc, about 0.00003 cc, or about 0.00001 cc.

In some embodiments, the collection device comprises a water vapor transmission rate of approximately 0.0005 grams or less (g) per 100 square inches per day at 90% relative humidity and 40 degrees Celsius. In some embodiments, a water vapor transmission rate of approximately 0.0005 g or less ensures that the gaseous sample is not contaminated or that the gaseous sample is not reduced during handling or transportation of the collection bag.

In some embodiments, the collection device comprises a water vapor transmission rate of about 0.00001 g to about 0.0005 g. In some embodiments, the collection device comprises a water vapor transmission rate of about 0.0005 g to about 0.0003 g, about 0.0005 g to about 0.0001 g, about 0.0005 g to about 0.00005 g, about 0.0005 g to about 0.00003 g, about 0.0005 g to about 0.00001 g, about 0.0003 g to about 0.0001 g, about 0.0003 g to about 0.00005 g, about 0.0003 g to about 0.00003 g, about 0.0003 g to about 0.00001 g, about 0.0001 g to about 0.00005 g, about 0.0001 g to about 0.00003 g, about 0.0001 g to about 0.00001 g, about 0.00005 g to about 0.00003 g, about 0.00005 g to about 0.00001 g, or about 0.00003 g to about 0.00001 g. In some embodiments, the collection device comprises a water vapor transmission rate of about 0.0005 g, about 0.0003 g, about 0.0001 g, about 0.00005 g, about 0.00003 g, or about 0.00001 g. In some embodiments, the collection device comprises a water vapor transmission rate of at most about 0.0003 g, about 0.0001 g, about 0.00005 g, about 0.00003 g, or about 0.00001 g.

In some embodiments, the collection bag is provided to retain a gaseous sample without affecting gas levels for at least 5 days. A retention of at least 5 days may ensure enough time for handling and transportation of a gaseous sample to before being analyzed by a testing facility.

In some embodiments, the collection bag is provided to retain a gaseous sample without affecting test results for about 2 days to about 20 days. In some embodiments, the collection bag is provided to retain a gaseous sample without affecting test results for at least two days. In some embodiments, the collection bag is provided to retain a gaseous sample without affecting test results for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag is provided to retain a gaseous sample without affecting test results for about 2 days, about 3 days, about 4 days, about 5 days, about 6 days, about 7 days, about 10 days, or about 20 days. In some embodiments, the collection bag is provided to retain a gaseous sample without affecting test results for at least about 2 days, about 3 days, about 4 days, about 5 days, about 6 days, about 7 days, or about 10 days.

In some embodiments, the collection bag is provided to retain carbon dioxide, hydrogen, methane, hydrogen sulfide, or combinations thereof without affecting test results for about 2 days to about 20 days. In some embodiments, the collection bag is provided to retain carbon dioxide, hydrogen, methane, hydrogen sulfide, or combinations thereof without affecting test results for at least two days. In some embodiments, the collection bag is provided to retain carbon dioxide, hydrogen, methane, hydrogen sulfide, or combinations thereof without affecting test results for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag is provided to retain carbon dioxide, hydrogen, methane, hydrogen sulfide, or combinations thereof without affecting test results for about 2 days, about 3 days, about 4 days, about 5 days, about 6 days, about 7 days, about 10 days, or about 20 days. In some embodiments, the collection bag is provided to retain carbon dioxide, hydrogen, methane, hydrogen sulfide, or combinations thereof without affecting test results for at least about 2 days, about 3 days, about 4 days, about 5 days, about 6 days, about 7 days, or about 10 days.

In some embodiments, the collection bag keeps the gaseous sample composition stable for about 2 days to about 20 days. In some embodiments, the collection bag stabilizes the gaseous sample for at least two days. In some embodiments, the collection bag stabilizes the gaseous sample for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes the gaseous sample for about 2 days, about 3 days, about 4 days, about 5 days, about 6 days, about 7 days, about 10 days, or about 20 days. In some embodiments, the collection bag stabilizes the gaseous sample for at least about 2 days, about 3 days, about 4 days, about 5 days, about 6 days, about 7 days, or about 10 days.

In some embodiments, the collection bag stabilizes carbon dioxide, hydrogen, methane, hydrogen sulfide, or combinations thereof for about 2 days to about 20 days. In some embodiments, the collection bag stabilizes carbon dioxide, hydrogen, methane, hydrogen sulfide, or combinations thereof for at least two days. In some embodiments, the collection bag stabilizes carbon dioxide, hydrogen, methane, hydrogen sulfide, or combinations thereof for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes retain carbon dioxide, hydrogen, methane, hydrogen sulfide, or combinations thereof for about 2 days, about 3 days, about 4 days, about 5 days, about 6 days, about 7 days, about 10 days, or about 20 days. In some embodiments, the collection bag stabilizes retain carbon dioxide, hydrogen, methane, hydrogen sulfide, or combinations thereof for at least about 2 days, about 3 days, about 4 days, about 5 days, about 6 days, about 7 days, or about 10 days.

In some embodiments, the collection bag stabilizes carbon dioxide for about 2 days to about 20 days. In some embodiments, the collection bag stabilizes carbon dioxide for at least two days. In some embodiments, the collection bag stabilizes carbon dioxide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide for about 2 days, about 3 days, about 4 days, about 5 days, about 6 days, about 7 days, about 10 days, or about 20 days. In some embodiments, the collection bag stabilizes carbon dioxide for at least about 2 days, about 3 days, about 4 days, about 5 days, about 6 days, about 7 days, or about 10 days.

In some embodiments, the collection bag stabilizes hydrogen for about 2 days to about 20 days. In some embodiments, the collection bag stabilizes hydrogen for at least two days. In some embodiments, the collection bag stabilizes hydrogen for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes hydrogen for about 2 days, about 3 days, about 4 days, about 5 days, about 6 days, about 7 days, about 10 days, or about 20 days. In some embodiments, the collection bag stabilizes hydrogen for at least about 2 days, about 3 days, about 4 days, about 5 days, about 6 days, about 7 days, or about 10 days.

In some embodiments, the collection bag stabilizes methane for about 2 days to about 20 days. In some embodiments, the collection bag stabilizes methane for at least two days. In some embodiments, the collection bag stabilizes methane for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes methane for about 2 days, about 3 days, about 4 days, about 5 days, about 6 days, about 7 days, about 10 days, or about 20 days. In some embodiments, the collection bag stabilizes methane for at least about 2 days, about 3 days, about 4 days, about 5 days, about 6 days, about 7 days, or about 10 days.

In some embodiments, the collection bag stabilizes hydrogen sulfide for about 2 days to about 20 days. In some embodiments, the collection bag stabilizes hydrogen sulfide for at least two days. In some embodiments, the collection bag stabilizes hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes hydrogen sulfide for about 2 days, about 3 days, about 4 days, about 5 days, about 6 days, about 7 days, about 10 days, or about 20 days. In some embodiments, the collection bag stabilizes hydrogen sulfide for at least about 2 days, about 3 days, about 4 days, about 5 days, about 6 days, about 7 days, or about 10 days.

In some embodiments, the collection bag stabilizes carbon dioxide and hydrogen for about 2 days to about 20 days. In some embodiments, the collection bag stabilizes carbon dioxide and hydrogen for at least two days. In some embodiments, the collection bag stabilizes carbon dioxide and hydrogen for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide and hydrogen for about 2 days, about 3 days, about 4 days, about 5 days, about 6 days, about 7 days, about 10 days, or about 20 days. In some embodiments, the collection bag stabilizes carbon dioxide and hydrogen for at least about 2 days, about 3 days, about 4 days, about 5 days, about 6 days, about 7 days, or about 10 days.

In some embodiments, the collection bag stabilizes carbon dioxide and methane for about 2 days to about 20 days. In some embodiments, the collection bag stabilizes carbon dioxide and methane for at least two days. In some embodiments, the collection bag stabilizes carbon dioxide and methane for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide and methane for about 2 days, about 3 days, about 4 days, about 5 days, about 6 days, about 7 days, about 10 days, or about 20 days. In some embodiments, the collection bag stabilizes carbon dioxide and methane for at least about 2 days, about 3 days, about 4 days, about 5 days, about 6 days, about 7 days, or about 10 days.

In some embodiments, the collection bag stabilizes carbon dioxide and hydrogen sulfide for about 2 days to about 20 days. In some embodiments, the collection bag stabilizes carbon dioxide and hydrogen sulfide for at least two days. In some embodiments, the collection bag stabilizes carbon dioxide and hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide and hydrogen sulfide for about 2 days, about 3 days, about 4 days, about 5 days, about 6 days, about 7 days, about 10 days, or about 20 days. In some embodiments, the collection bag stabilizes carbon dioxide and hydrogen sulfide for at least about 2 days, about 3 days, about 4 days, about 5 days, about 6 days, about 7 days, or about 10 days.

In some embodiments, the collection bag stabilizes hydrogen and methane for about 2 days to about 20 days. In some embodiments, the collection bag stabilizes hydrogen and methane for at least two days. In some embodiments, the collection bag stabilizes hydrogen and methane for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes hydrogen and methane for about 2 days, about 3 days, about 4 days, about 5 days, about 6 days, about 7 days, about 10 days, or about 20 days. In some embodiments, the collection bag stabilizes hydrogen and methane for at least about 2 days, about 3 days, about 4 days, about 5 days, about 6 days, about 7 days, or about 10 days.

In some embodiments, the collection bag stabilizes hydrogen and hydrogen sulfide for about 2 days to about 20 days. In some embodiments, the collection bag stabilizes hydrogen and hydrogen sulfide for at least two days. In some embodiments, the collection bag stabilizes hydrogen and hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes hydrogen and hydrogen sulfide for about 2 days, about 3 days, about 4 days, about 5 days, about 6 days, about 7 days, about 10 days, or about 20 days. In some embodiments, the collection bag stabilizes hydrogen and hydrogen sulfide for at least about 2 days, about 3 days, about 4 days, about 5 days, about 6 days, about 7 days, or about 10 days.

In some embodiments, the collection bag stabilizes methane and hydrogen sulfide for about 2 days to about 20 days. In some embodiments, the collection bag stabilizes methane and hydrogen sulfide for at least two days. In some embodiments, the collection bag stabilizes methane and hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes methane and hydrogen sulfide for about 2 days, about 3 days, about 4 days, about 5 days, about 6 days, about 7 days, about 10 days, or about 20 days. In some embodiments, the collection bag stabilizes methane and hydrogen sulfide for at least about 2 days, about 3 days, about 4 days, about 5 days, about 6 days, about 7 days, or about 10 days.

In some embodiments, the collection bag stabilizes carbon dioxide, hydrogen, and methane for about 2 days to about 20 days. In some embodiments, the collection bag stabilizes carbon dioxide, hydrogen, and methane for at least two days. In some embodiments, the collection bag stabilizes carbon dioxide, hydrogen, and methane for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide, hydrogen, and methane for about 2 days, about 3 days, about 4 days, about 5 days, about 6 days, about 7 days, about 10 days, or about 20 days. In some embodiments, the collection bag stabilizes carbon dioxide, hydrogen, and methane for at least about 2 days, about 3 days, about 4 days, about 5 days, about 6 days, about 7 days, or about 10 days.

In some embodiments, the collection bag stabilizes carbon dioxide, hydrogen, and hydrogen sulfide for about 2 days to about 20 days. In some embodiments, the collection bag stabilizes carbon dioxide, hydrogen, and hydrogen sulfide for at least two days. In some embodiments, the collection bag stabilizes carbon dioxide, hydrogen, and hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide, hydrogen, and hydrogen sulfide for about 2 days, about 3 days, about 4 days, about 5 days, about 6 days, about 7 days, about 10 days, or about 20 days. In some embodiments, the collection bag stabilizes carbon dioxide, hydrogen, and hydrogen sulfide for at least about 2 days, about 3 days, about 4 days, about 5 days, about 6 days, about 7 days, or about 10 days.

In some embodiments, the collection bag stabilizes methane, hydrogen, and hydrogen sulfide for about 2 days to about 20 days. In some embodiments, the collection bag stabilizes methane, hydrogen, and hydrogen sulfide for at least two days. In some embodiments, the collection bag stabilizes methane, hydrogen, and hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes methane, hydrogen, and hydrogen sulfide for about 2 days, about 3 days, about 4 days, about 5 days, about 6 days, about 7 days, about 10 days, or about 20 days. In some embodiments, the collection bag stabilizes methane, hydrogen, and hydrogen sulfide for at least about 2 days, about 3 days, about 4 days, about 5 days, about 6 days, about 7 days, or about 10 days.

In some embodiments, the collection bag stabilizes carbon dioxide, methane, hydrogen, and hydrogen sulfide for about 2 days to about 20 days. In some embodiments, the collection bag stabilizes carbon dioxide, methane, hydrogen, and hydrogen sulfide for at least two days. In some embodiments, the collection bag stabilizes carbon dioxide, methane, hydrogen, and hydrogen sulfide for about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the collection bag stabilizes carbon dioxide, methane, hydrogen, and hydrogen sulfide for about 2 days, about 3 days, about 4 days, about 5 days, about 6 days, about 7 days, about 10 days, or about 20 days. In some embodiments, the collection bag stabilizes carbon dioxide, methane, hydrogen, and hydrogen sulfide for at least about 2 days, about 3 days, about 4 days, about 5 days, about 6 days, about 7 days, or about 10 days.

In some embodiments, the collection bag retains at least or about 70%, 75%, 80%, 85%, 90%, 95%, 99%, or more than 99% of the gaseous sample after at least two days of being collected. In some embodiments, the collection bag retains at least or about 70%, 75%, 80%, 85%, 90%, 95%, 99%, or more than 99% of the gaseous sample after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the gaseous sample after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the gas retained is percentage by weight (wt. %). In some embodiments, the percentage of the gas retained is compared to the amount (e.g., parts per million or ppm) or concentration of gas at time 0 (e.g., Day 0).

In some embodiments, the collection bag retains at least or about 70%, 75%, 80%, 85%, 90%, 95%, 99%, or more than 99% of the carbon dioxide, hydrogen, methane, hydrogen sulfide, or combinations thereof after at least two days of being collected. In some embodiments, the collection bag retains at least or about 70%, 75%, 80%, 85%, 90%, 95%, 99%, or more than 99% of the carbon dioxide, hydrogen, methane, hydrogen sulfide, or combinations thereof after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide, hydrogen, methane, hydrogen sulfide, or combinations thereof after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide, hydrogen, methane, hydrogen sulfide, or combinations thereof retained is percentage by weight (wt. %). In some embodiments, the percentage of the carbon dioxide, hydrogen, methane, hydrogen sulfide, or combinations thereof retained is compared to the amount (e.g., ppm) or concentration of the carbon dioxide, hydrogen, methane, hydrogen sulfide, or combinations thereof at time 0 (e.g., Day 0).

In some embodiments, the collection bag retains at least or about 70%, 75%, 80%, 85%, 90%, 95%, 99%, or more than 99% of the carbon dioxide after at least two days of being collected. In some embodiments, the collection bag retains at least or about 70%, 75%, 80%, 85%, 90%, 95%, 99%, or more than 99% of the carbon dioxide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide, retained is percentage by weight (wt. %). In some embodiments, the percentage of the carbon dioxide retained is compared to the amount (e.g., ppm) or concentration of the carbon dioxide at time 0 (e.g., Day 0).

In some embodiments, the collection bag retains at least or about 70%, 75%, 80%, 85%, 90%, 95%, 99%, or more than 99% of the hydrogen after at least two days of being collected. In some embodiments, the collection bag retains at least or about 70%, 75%, 80%, 85%, 90%, 95%, 99%, or more than 99% of the hydrogen after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the hydrogen after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the hydrogen retained is percentage by weight (wt. %). In some embodiments, the percentage of the hydrogen retained is compared to the amount (e.g., ppm) or concentration of the hydrogen at time 0 (e.g., Day 0).

In some embodiments, the collection bag retains at least or about 70%, 75%, 80%, 85%, 90%, 95%, 99%, or more than 99% of the methane after at least two days of being collected. In some embodiments, the collection bag retains at least or about 70%, 75%, 80%, 85%, 90%, 95%, 99%, or more than 99% of the methane after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the methane after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the methane retained is percentage by weight (wt. %). In some embodiments, the percentage of the methane retained is compared to the amount (e.g., ppm) or concentration of the methane at time 0 (e.g., Day 0).

In some embodiments, the collection bag retains at least or about 70%, 75%, 80%, 85%, 90%, 95%, 99%, or more than 99% of the hydrogen sulfide after at least two days of being collected. In some embodiments, the collection bag retains at least or about 70%, 75%, 80%, 85%, 90%, 95%, 99%, or more than 99% of the hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the hydrogen sulfide retained is percentage by weight (wt. %). In some embodiments, the percentage of the hydrogen sulfide retained is compared to the amount (e.g., ppm) or concentration of the hydrogen sulfide at time 0 (e.g., Day 0).

In some embodiments, the collection bag retains at least or about 70%, 75%, 80%, 85%, 90%, 95%, 99%, or more than 99% of the carbon dioxide and methane after at least two days of being collected. In some embodiments, the collection bag retains at least or about 70%, 75%, 80%, 85%, 90%, 95%, 99%, or more than 99% of the carbon dioxide and methane after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide and methane after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide and methane retained is percentage by weight (wt. %). In some embodiments, the percentage of the carbon dioxide and methane retained is compared to the amount (e.g., ppm) or concentration of the carbon dioxide and methane at time 0 (e.g., Day 0).

In some embodiments, the collection bag retains at least or about 70%, 75%, 80%, 85%, 90%, 95%, 99%, or more than 99% of the carbon dioxide and hydrogen after at least two days of being collected. In some embodiments, the collection bag retains at least or about 70%, 75%, 80%, 85%, 90%, 95%, 99%, or more than 99% of the carbon dioxide and hydrogen after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide and hydrogen after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide and hydrogen retained is percentage by weight (wt. %). In some embodiments, the percentage of the carbon dioxide and hydrogen retained is compared to the amount (e.g., ppm) or concentration of the carbon dioxide and hydrogen at time 0 (e.g., Day 0).

In some embodiments, the collection bag retains at least or about 70%, 75%, 80%, 85%, 90%, 95%, 99%, or more than 99% of the carbon dioxide and hydrogen sulfide after at least two days of being collected. In some embodiments, the collection bag retains at least or about 70%, 75%, 80%, 85%, 90%, 95%, 99%, or more than 99% of the carbon dioxide and hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide and hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide and hydrogen sulfide retained is percentage by weight (wt. %). In some embodiments, the percentage of the carbon dioxide and hydrogen sulfide retained is compared to the amount (e.g., ppm) or concentration of the carbon dioxide and hydrogen sulfide at time 0 (e.g., Day 0).

In some embodiments, the collection bag retains at least or about 70%, 75%, 80%, 85%, 90%, 95%, 99%, or more than 99% of the methane and hydrogen after at least two days of being collected. In some embodiments, the collection bag retains at least or about 70%, 75%, 80%, 85%, 90%, 95%, 99%, or more than 99% of the methane and hydrogen after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the methane and hydrogen after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the methane and hydrogen retained is percentage by weight (wt. %). In some embodiments, the percentage of the methane and hydrogen retained is compared to the amount (e.g., ppm) or concentration of the methane and hydrogen at time 0 (e.g., Day 0).

In some embodiments, the collection bag retains at least or about 70%, 75%, 80%, 85%, 90%, 95%, 99%, or more than 99% of the methane and hydrogen sulfide after at least two days of being collected. In some embodiments, the collection bag retains at least or about 70%, 75%, 80%, 85%, 90%, 95%, 99%, or more than 99% of the methane and hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the methane and hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the methane and hydrogen sulfide retained is percentage by weight (wt. %). In some embodiments, the percentage of the methane and hydrogen sulfide retained is compared to the amount (e.g., ppm) or concentration of the methane and hydrogen sulfide at time 0 (e.g., Day 0).

In some embodiments, the collection bag retains at least or about 70%, 75%, 80%, 85%, 90%, 95%, 99%, or more than 99% of the hydrogen and hydrogen sulfide after at least two days of being collected. In some embodiments, the collection bag retains at least or about 70%, 75%, 80%, 85%, 90%, 95%, 99%, or more than 99% of the hydrogen and hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the hydrogen and hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the hydrogen and hydrogen sulfide retained is percentage by weight (wt. %). In some embodiments, the percentage of the hydrogen and hydrogen sulfide retained is compared to the amount (e.g., ppm) or concentration of the hydrogen and hydrogen sulfide at time 0 (e.g., Day 0).

In some embodiments, the collection bag retains at least or about 70%, 75%, 80%, 85%, 90%, 95%, 99%, or more than 99% of the carbon dioxide, methane, and hydrogen after at least two days of being collected. In some embodiments, the collection bag retains at least or about 70%, 75%, 80%, 85%, 90%, 95%, 99%, or more than 99% of the carbon dioxide, methane, and hydrogen after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide, methane, and hydrogen after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide, methane, and hydrogen retained is percentage by weight (wt. %). In some embodiments, the percentage of the carbon dioxide, methane, and hydrogen retained is compared to the amount (e.g., ppm) or concentration of the carbon dioxide, methane, and hydrogen at time 0 (e.g., Day 0).

In some embodiments, the collection bag retains at least or about 70%, 75%, 80%, 85%, 90%, 95%, 99%, or more than 99% of the carbon dioxide, methane, and hydrogen sulfide after at least two days of being collected. In some embodiments, the collection bag retains at least or about 70%, 75%, 80%, 85%, 90%, 95%, 99%, or more than 99% of the carbon dioxide, methane, and hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide, methane, and hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide, methane, and hydrogen sulfide retained is percentage by weight (wt. %). In some embodiments, the percentage of the carbon dioxide, methane, and hydrogen sulfide retained is compared to the amount (e.g., ppm) or concentration of the carbon dioxide, methane, and hydrogen sulfide at time 0 (e.g., Day 0).

In some embodiments, the collection bag retains at least or about 70%, 75%, 80%, 85%, 90%, 95%, 99%, or more than 99% of the methane, hydrogen, and hydrogen sulfide after at least two days of being collected. In some embodiments, the collection bag retains at least or about 70%, 75%, 80%, 85%, 90%, 95%, 99%, or more than 99% of the methane, hydrogen, and hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the methane, hydrogen, and hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the methane, hydrogen, and hydrogen sulfide retained is percentage by weight (wt. %). In some embodiments, the percentage of the methane, hydrogen, and hydrogen sulfide retained is compared to the amount (e.g., ppm) or concentration of the methane, hydrogen, and hydrogen sulfide at time 0 (e.g., Day 0).

In some embodiments, the collection bag retains at least or about 70%, 75%, 80%, 85%, 90%, 95%, 99%, or more than 99% of the carbon dioxide, methane, hydrogen, and hydrogen sulfide after at least two days of being collected. In some embodiments, the collection bag retains at least or about 70%, 75%, 80%, 85%, 90%, 95%, 99%, or more than 99% of the carbon dioxide, methane, hydrogen, and hydrogen sulfide after at least or about 2 days, 3 days, 4 days, 5 days, 6 days, 7 days, 10 days, or 20 days of being collected. In some embodiments, the collection bag retains at least or about 70%, 75%, 80%, 85%, 90%, 95%, 99%, or more than 99% of the carbon dioxide, methane, hydrogen, and hydrogen sulfide after about 2 days to about 3 days, about 2 days to about 4 days, about 2 days to about 5 days, about 2 days to about 6 days, about 2 days to about 7 days, about 2 days to about 10 days, about 2 days to about 20 days, about 3 days to about 4 days, about 3 days to about 5 days, about 3 days to about 6 days, about 3 days to about 7 days, about 3 days to about 10 days, about 3 days to about 20 days, about 4 days to about 5 days, about 4 days to about 6 days, about 4 days to about 7 days, about 4 days to about 10 days, about 4 days to about 20 days, about 5 days to about 6 days, about 5 days to about 7 days, about 5 days to about 10 days, about 5 days to about 20 days, about 6 days to about 7 days, about 6 days to about 10 days, about 6 days to about 20 days, about 7 days to about 10 days, about 7 days to about 20 days, or about 10 days to about 20 days. In some embodiments, the percentage of the carbon dioxide, methane, hydrogen, and hydrogen sulfide retained is percentage by weight (wt. %). In some embodiments, the percentage of the carbon dioxide, methane, hydrogen, and hydrogen sulfide retained is compared to the amount (e.g., ppm) or concentration of the carbon dioxide, methane, hydrogen, and hydrogen sulfide at time 0 (e.g., Day 0).

### Mouthpiece

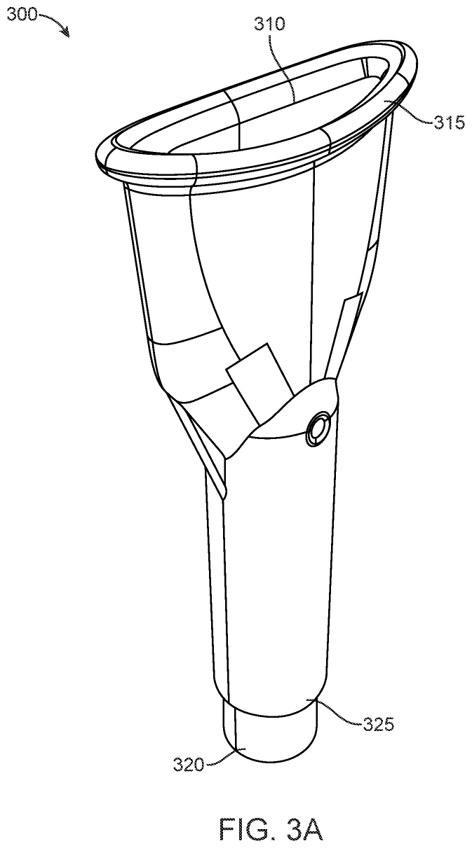

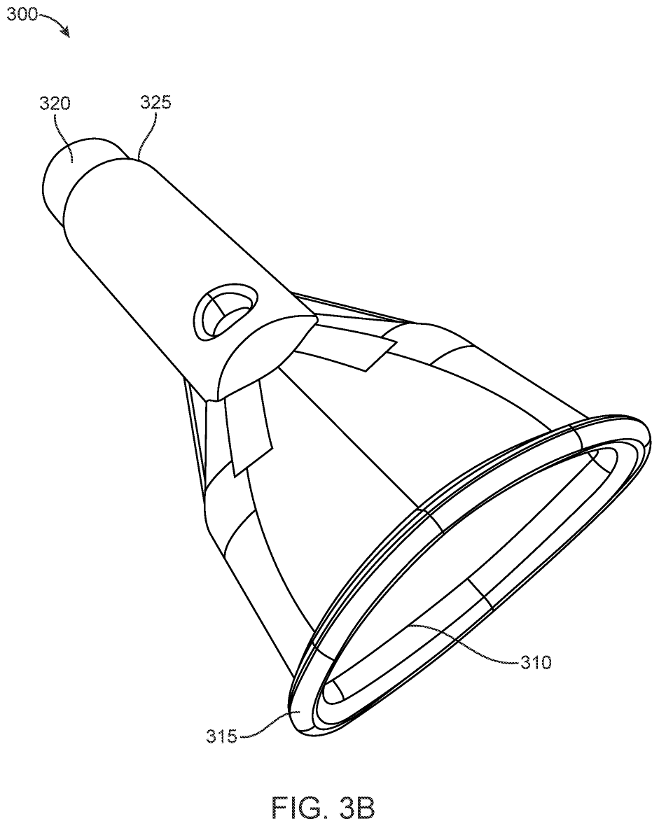

In some embodiments, microbiome sample collection systems and devices include a mouthpiece to facilitate acquisition of a gaseous sample from a subject. FIGS. 3A and 3B depict a mouthpiece component 300, according to some embodiments. In some embodiments, the mouthpiece 300 comprises a proximal end 310 to receive a mouth of a subject and a distal end 320 to for attachment to a collection bag. In some embodiments, the mouthpiece 300 is utilized to convey a gaseous sample from a subject into a collection bag.

In some the proximal end 310 of the mouthpiece 300 comprises a lip 315. In some embodiments, a subject places his/her lips over lip 315. The lip 315 may facilitate retention of the mouthpiece 300 within the subject's mouth. In some embodiments, the distal end 310 of the mouthpiece is substantially elliptical. The shape of the mouthpiece 300 may facilitate proper transmission of the gaseous sample from the subject to a collection bag connected to a distal end 320 of the mouthpiece.

In some embodiments, the distal end 320 of the mouthpiece 300 is configured to be received by a valve of a collection bag (e.g. valve 150 of collection bag 100 as shown in FIG. 1). In some embodiments, the distal end 320 forms an interference or close tolerance fit with the valve, such that the mouthpiece distal end of the mouthpiece is retained by the valve. In some embodiments, the outer circumference of the distal end 320 is approximately equal to or slightly larger than the inner circumference of the valve to form an interference type fit. In some embodiments, the diameter forming the circumference of the distal end 320 of the mouthpiece is approximately 0.375 inches.

In some embodiments, the distal end 320 comprises a lip 325 which has a diameter slightly larger than the diameter of the portion of the distal end to be received by a valve of a collection bag. In some embodiments, the lip 325 limits the depth to which the distal end 320 of the mouthpiece 300 is inserted into the valve. In some embodiments, the lip 325 prevents interference with a seal which creates the one-way valve. In some embodiments, insertion of the mouthpiece opens a seal to an interior space of the collection bag. In some embodiments, removal of the mouthpiece facilitates closure of a seal to an interior space of the collection bag.

In some embodiments, the proximal end 310 tapers inward toward the distal end 325. In some embodiments, tapering of the mouthpiece provides a convergent nozzle to facilitate transmission of the gaseous sample from the patient into a collection bag.

### Methods of Use

Described herein are methods and devices for assessing a microbiome of an individual. In some embodiments, the method comprises a) collecting a sample from the individual using a device described herein; b) detecting one or more gases in the sample; and c) assessing the microbiome of the individual based on the detection of the one or more gases in the sample.

Various types of samples may be analyzed using the methods and devices as described herein. In some embodiments, the sample comprises a gas sample from the individual. In some embodiments, the sample comprises a breath sample from the individual.

Described herein are methods and devices for assessing a microbiome of an individual, wherein one or more gases are detected. In some embodiments, the one or more gases comprises carbon dioxide, hydrogen, methane, hydrogen sulfide, or combinations thereof. In some embodiments, at least one, two, three, four, five, six, or more than six gases are detected. In some embodiments, a first gas of the one or more gases is hydrogen sulfide. In some embodiments, a first gas of the one or more gases is carbon dioxide. In some embodiments, a first gas of the one or more gases is hydrogen. In some embodiments, a first gas of the one or more gases is methane.

In some embodiments, at least two gases are detected. In some embodiments, the at least two gases comprise carbon dioxide, hydrogen, methane, or hydrogen sulfide. In some embodiments, the at least two gases comprise carbon dioxide and hydrogen. In some embodiments, the at least two gases comprise carbon dioxide and methane. In some embodiments, the at least two gases comprise carbon dioxide and hydrogen sulfide. In some embodiments, the at least two gases comprise hydrogen and methane. In some embodiments, the at least two gases comprise hydrogen and hydrogen sulfide. In some embodiments, the at least two gases comprise methane and hydrogen sulfide.

In some embodiments, at least three gases are detected. In some embodiments, the at least three gases comprise carbon dioxide, hydrogen, methane, or hydrogen sulfide. In some embodiments, the at least three gases comprise carbon dioxide, hydrogen, and methane. In some embodiments, the at least three gases comprise carbon dioxide, hydrogen, and hydrogen sulfide. In some embodiments, the at least three gases comprise hydrogen, methane, and hydrogen sulfide.

In some embodiments, at least four gases are detected. In some embodiments, the at least four gases comprise carbon dioxide, hydrogen, methane, and hydrogen sulfide.

Described herein are methods and devices for assessing a microbiome of an individual, wherein the method further comprises determining whether an individual has a disease or disorder based on the detection of the one or more gases in the sample. In some embodiments, the one or more gases detected are associated with the disease or disorder.

In some embodiments, the disease or disorder is a metabolic disorder. In some embodiments, a disease or disorder is diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, glomerular disease, or intestinal methanogenic overgrowth. In some embodiments,

In some embodiments, the one or more gases is elevated above a threshold level of the one or more gases derived from a cohort of control samples. In some instances, the elevated level of the one or more gases provides an indication of the disease or disorder. In some instances, the one or more gases is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of the one or more gases derived from a cohort of control samples. In some embodiments, the one or more gases comprises carbon dioxide, hydrogen, methane, hydrogen sulfide, or combinations thereof.

In some instances, the level of the one or more gases is decreased below a threshold level of said one or more gases derived from a cohort of control samples. In some instances, the decreased level of the one or more gases provides an indication of the disease or disorder. In some instances, the one or more gases is decreased at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold below the threshold level of the one or more gases derived from a cohort of control samples. In some embodiments, the one or more gases comprises carbon dioxide, hydrogen, methane, hydrogen sulfide, or combinations thereof.

In some embodiments, the one or more gases is hydrogen and is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of hydrogen derived from a cohort of control samples. In some embodiments, the elevated level of hydrogen provides an indication of intestinal methanogenic overgrowth. In some embodiments, the individual has or is suspected of having intestinal methanogenic overgrowth when the hydrogen is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of hydrogen derived from a cohort of control samples.

In some embodiments, the one or more gases is methane and is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of methane derived from a cohort of control samples. In some embodiments, the elevated level of methane provides an indication of small intestinal bacterial overgrowth. In some embodiments, the individual has or is suspected of having small intestinal bacterial overgrowth when the methane is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of methane derived from a cohort of control samples. In some embodiments, a threshold level of methane for diagnosis of small intestinal bacterial overgrowth (SIBO) is about 10 parts per million (ppm). In some embodiments, a threshold level of methane for diagnosis of small intestinal bacterial overgrowth (SIBO) is about 5 ppm to about 30 ppm. In some embodiments, a threshold level of methane for diagnosis of small intestinal bacterial overgrowth (SIBO) is about 5 ppm to about 10 ppm, about 5 ppm to about 15 ppm, about 5 ppm to about 20 ppm, about 5 ppm to about 25 ppm, about 5 ppm to about 30 ppm, about 10 ppm to about 15 ppm, about 10 ppm to about 20 ppm, about 10 ppm to about 25 ppm, about 10 ppm to about 30 ppm, about 15 ppm to about 20 ppm, about 15 ppm to about 25 ppm, about 15 ppm to about 30 ppm, about 20 ppm to about 25 ppm, about 20 ppm to about 30 ppm, or about 25 ppm to about 30 ppm. In some embodiments, a threshold level of methane for diagnosis of small intestinal bacterial overgrowth (SIBO) is about 5 ppm, about 10 ppm, about 15 ppm, about 20 ppm, about 25 ppm, or about 30 ppm. In some embodiments, a threshold level of methane for diagnosis of small intestinal bacterial overgrowth (SIBO) is at least about 5 ppm, about 10 ppm, about 15 ppm, about 20 ppm, or about 25 ppm.

In some embodiments, the one or more gases is hydrogen sulfide and is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of hydrogen sulfide derived from a cohort of control samples. In some embodiments, the elevated level of hydrogen sulfide provides an indication of various diseases or disorders. In some embodiments, the individual has or is suspected of having various diseases or disorders when the hydrogen sulfide is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of hydrogen sulfide derived from a cohort of control samples. In some embodiments, the various diseases or disorders associated with elevated hydrogen sulfide include, but are not limited to, diarrhea, Crohn's disease, ulcerative colitis, and colorectal cancer. In some embodiments, using a collection and measurement system described herein, exhaled H₂S (hydrogen sulfide) levels are higher in patients with diarrhea as compared to healthy controls and patients with constipation. In some embodiments, H₂S measured during clinical breath testing is a biomarker for diarrhea.

In some embodiments, levels of the gases to be analyzed are calibrated based on the amount of time they reside within a collection bag. A collection bag may be labeled with the date and time at which the sample was provided into the bag. Based on the time at which the sample was provided, the level of gases to may be adjusted to a calibrated or expected level. The expected levels may be based on known deterioration, reduction, growth, or increase of the gases over a period of time. Based on the duration the gaseous samples reside within the bag, the levels may be adjusted by a percentage to arrive at an expected value at the time the sample was collected. In some embodiments, gaseous sample levels at the time of analysis are adjusted by about 0.5% to about 15%. In some embodiments, gaseous sample levels at the time of analysis are adjusted by about 0.5% to about 1%, about 0.5% to about 2%, about 0.5% to about 3%, about 0.5% to about 5%, about 0.5% to about 10%, about 0.5% to about 15%, about 1% to about 2%, about 1% to about 3%, about 1% to about 5%, about 1% to about 10%, about 1% to about 15%, about 2% to about 3%, about 2% to about 5%, about 2% to about 10%, about 2% to about 15%, about 3% to about 5%, about 3% to about 10%, about 3% to about 15%, about 5% to about 10%, about 5% to about 15%, or about 10% to about 15%. In some embodiments, gaseous sample levels at the time of analysis are adjusted by about 0.5%, about 1%, about 2%, about 3%, about 5%, about 10%, or about 15%. In some embodiments, gaseous sample levels at the time of analysis are adjusted by at least about 0.5%, about 1%, about 2%, about 3%, about 5%, or about 10%. In some embodiments, gaseous sample levels at the time of analysis are adjusted by at most about 1%, about 2%, about 3%, about 5%, about 10%, or about 15%.

Described herein are methods and devices for assessing a microbiome of an individual with improved sensitivity, specificity, reliability, and accuracy for determining whether an individual has a disease or disorder based on the detection of the one or more gases (e.g., CH₄, H₂S, and H₂) in the sample. In some instances, the one or more gases provide at least about an 80% predictive value as determined by receiver operating characteristic (ROC) curve or area under the curve (AUC) analysis in assessing the likelihood of a disease or disorder in the individual. In some instances, the one or more gases provide at least about an 65%, at least about 70%, at least about 75%, at least about 80%, at least about 85%, at least about 90%, at least about 95%, or more than about 95% predictive value as determined by ROC curve or AUC curve analysis in assessing the diseases or disorder in the individual. In some instances, the predictive value is a positive predictive value. In some instances, the predictive value is a negative predictive value. In some instances, the disease or disorder is a metabolic disorder. In some instances, the disease or disorder is diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, glomerular disease, or intestinal methanogenic overgrowth.

The one or more gases (e.g., CH₄, H₂S, and H₂) described herein may provide a sensitivity of at least about 80% as determined by ROC curve or AUC curve analysis in assessing the likelihood of a diseases or disorder in the individual. In some instances, the one or more gases provide a sensitivity of at least about 65%, at least about 70%, at least about 75%, at least about 80%, at least about 85%, at least about 90%, at least about 95%, or more than about 95% as determined by ROC curve or AUC curve analysis in assessing the likelihood of a diseases or disorder in the individual. In some instances, the sensitivity is compared to an individual without the disease or disorder. In some instances, the sensitivity is compared to an individual with a milder disease or disorder. In some instances, the disease or disorder is a metabolic disorder. In some instances, the disease or disorder is diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, glomerular disease, or intestinal methanogenic overgrowth.

The one or more gases (e.g., CH₄, H₂S, and H₂) described herein may provide an accuracy of at least about 65%, at least about 70%, at least about 75%, at least about 80%, at least about 85%, at least about 90%, at least about 95%, or more than about 95% as determined by ROC curve or AUC curve analysis in assessing the likelihood of a diseases or disorder in the individual. In some instances, the accuracy is compared to an individual without the disease or disorder. In some instances, the accuracy is compared to an individual with a milder disease or disorder. In some instances, the disease or disorder is a metabolic disorder. In some instances, the disease or disorder is diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, glomerular disease, or intestinal methanogenic overgrowth.

### Kits

In some embodiments, a kit is provided comprising a plurality of collection bags and one or more mouthpiece components, as described herein. In some embodiments, the kit provides a plurality of collection bags such at least 2, 3, 4, 5, 6, 7, 8, 9, 10, or more than 10 samples are collected. In some embodiments, the kit provides a plurality of collection bags such at least 9 samples are collected. In some embodiments, the kit provides at least 1, 2, 3, 4, 5, 6, 7, 8, 9, 10, 12, 14, 16, or more than 16 collection bags. In some embodiments, the kit further comprises instructions detailing how to provide a sample using the kit. In some embodiments, the kit further comprises shipping materials for shipping a plurality of collection bags containing gaseous samples within.

In some embodiments, a sampling kit comprises a sugar solution or drink to be consumed by the subject prior to producing gaseous samples. In some embodiments, the solution or drink consumed by the subject comprises a sugar that is fermented by bacteria of the individual's microbiome. In some embodiments, one or more gases are produced by fermentation by the bacteria. In some embodiments, the one or more gases comprises carbon dioxide, hydrogen, methane, hydrogen sulfide, or combinations thereof.

In some embodiments, the sampling kit comprises fewer mouthpieces than collection bags, as the mouthpieces are removably attachable to the collection bag and a single mouthpiece is used more than once for sample collection. In some embodiment, mouthpieces are removed from the collection bags prior to shipping to reduce the size of the collection bags.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, kits comprise one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included.

In some embodiments, the instructions prescribe a predetermined amount of time that a subject should wait between providing a gaseous samples into collection bags. In some embodiments, the a subject is instructed to wait at least 15 minutes between providing gaseous samples into the collection bags. In some embodiments, the duration which the subject should wait before providing a subsequent sample into a collection bag is about 5 minutes to about 30 minutes. In some embodiments, the duration which the subject should wait before providing a subsequent sample into a collection bag is about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes. In some embodiments, the duration which the subject should wait before providing a subsequent sample into a collection bag is about 5 minutes, about 10 minutes, about 15 minutes, about 20 minutes, about 25 minutes, or about 30 minutes. In some embodiments, the duration which the subject should wait before providing a subsequent sample into a collection bag is at least about 5 minutes, about 10 minutes, about 15 minutes, about 20 minutes, or about 25 minutes. In some embodiments, the duration which the subject should wait before providing a subsequent sample into a collection bag is at most about 10 minutes, about 15 minutes, about 20 minutes, about 25 minutes, or about 30 minutes.

In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. In some embodiments, a label also indicates directions for use of the contents, such as in the methods described herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### NUMBERED EMBODIMENTS

Numbered embodiment 1 comprises a device for collecting a microbiome-derived gaseous sample, comprising: a multilayer composite forming a bag having an interior space for containing the microbiome-derived gaseous sample, the multilayer composite comprising: an inner layer comprising a first polymer film, a second layer, adjacent to the inner layer, comprising a foil, a third layer, adjacent to the second layer, comprising a second polymer film, and an exterior layer, adjacent to the third layer; and a valve for in fluid connection with the interior space of the bag, wherein the valve is a one-way valve. Numbered embodiment 2 comprises a device of numbered embodiment 1, wherein the first polymer film comprises low-density polyethylene. Numbered embodiment 3 comprises a device of any one of numbered embodiments 1-2, wherein the first polymer film comprises linear low-density polyethylene. Numbered embodiment 4 comprises a device of any one of numbered embodiments 1-3, wherein the foil is an aluminum foil. Numbered embodiment 5 comprises a device of any one of numbered embodiments 1-4, wherein the inner layer and the second layer comprise a metalized film. Numbered embodiment 6 comprises a device of any one of numbered embodiments 1-5, wherein the metalized film comprises biaxially-oriented polyethylene terephthalate. Numbered embodiment 7 comprises a device of any one of numbered embodiments 1-6, wherein the foil is an aluminum foil. Numbered embodiment 8 comprises a device of any one of numbered embodiments 1-7, wherein the third layer and the second layer comprise a metalized film. Numbered embodiment 9 comprises a device of any one of numbered embodiments 1-8, wherein the metalized film comprises biaxially-oriented polyethylene terephthalate. Numbered embodiment 10 comprises a device of any one of numbered embodiments 1-9, wherein the exterior layer comprises nylon. Numbered embodiment 11 comprises a device of any one of numbered embodiments 1-10, wherein the exterior layer comprises a biaxially oriented nylon film (BON). Numbered embodiment 12 comprises a device of any one of numbered embodiments 1-11, further comprising a mouthpiece. Numbered embodiment 13 comprises a device of any one of numbered embodiments 1-12, wherein the exterior layer is approximately 0.0006 inches thick. Numbered embodiment 14 comprises a device of any one of numbered embodiments 1-13, wherein the interior layer is approximately 0.00225 inches thick. Numbered embodiment 15 comprises a device of any one of numbered embodiments 1-14, wherein the second layer is approximately 0.0003 inches thick. Numbered embodiment 16 comprises a device of any one of numbered embodiments 1-15, wherein the third layer is approximately 0.00015 inches thick. Numbered embodiment 17 comprises a device of any one of numbered embodiments 1-16, wherein the bag is a flat bag. Numbered embodiment 18 comprises a device of any one of numbered embodiments 1-17, wherein the bag is formed by two layers of the multilayer composite, and wherein the interior space is formed by a seal between the two layers. Numbered embodiment 19 comprises a device of any one of numbered embodiments 1-18, wherein the seal has a width of approximately 0.375 inches. Numbered embodiment 20 comprises a device of any one of numbered embodiments 1-19, wherein the seal is formed by heat sealing. Numbered embodiment 21 comprises a device of any one of numbered embodiments 1-20, wherein the heat sealing comprises applying a temperature of approximately 400 degrees Fahrenheit and a pressure of approximately 40 pounds per square inch for a dwell time of approximately 1.5 seconds to form the seal. Numbered embodiment 22 comprises a device of any one of numbered embodiments 1-21, wherein the interior space comprises a width of approximately 4 inches, and a length of approximately 6 inches. Numbered embodiment 23 comprises a device of any one of numbered embodiments 1-22, wherein an aperture having a diameter of approximately 0.375 inches is formed through one layer of the multilayer composite to receive the valve. Numbered embodiment 24 comprises a device of any one of numbered embodiments 1-23, wherein the aperture is located approximately 1.25 inches from a side of the seal which forms the width of the interior space, and wherein the aperture is located approximately 2 inches from a side of the seal which forms the length of the interior space. Numbered embodiment 25 comprises a device of any one of numbered embodiments 1-24, wherein the device comprises a seal strength of approximately 18 pounds per inch. Numbered embodiment 26 comprises a device of any one of numbered embodiments 1-25, wherein the device comprises a tensile strength of approximately 19 pounds per inch. Numbered embodiment 27 comprises a device of any one of numbered embodiments 1-26, wherein the device comprises a puncture resistance of approximately 20 pounds. Numbered embodiment 28 comprises a device of any one of numbered embodiments 1-27, wherein the device comprises an oxygen transmission rate of approximately 0.0005 cubic centimeters per 100 square inches per day at 0% relative humidity and 23 degrees Celsius. Numbered embodiment 29 comprises a device of any one of numbered embodiments 1-28, wherein the device comprises a water vapor transmission rate of approximately 0.0005 grams per 100 square inches per day at 90% relative humidity and 40 degrees Celsius. Numbered embodiment 30 comprises a device of any one of numbered embodiments 1-29, wherein the microbiome-derived gaseous sample is a breath sample. Numbered embodiment 31 comprises a device of any one of numbered embodiments 1-30, wherein the microbiome-derived gaseous sample comprises one or more gases. Numbered embodiment 32 comprises a device of any one of numbered embodiments 1-31, wherein the one or more gases comprises carbon dioxide, hydrogen, methane, hydrogen sulfide, or combinations thereof. Numbered embodiment 33 comprises a device of any one of numbered embodiments 1-32, wherein a first gas of the one or more gases is hydrogen sulfide. Numbered embodiment 34 comprises a device of any one of numbered embodiments 1-33, wherein the one or more gases comprises carbon dioxide and hydrogen sulfide. Numbered embodiment 35 comprises a device of any one of numbered embodiments 1-34, wherein the one or more gases comprises hydrogen and hydrogen sulfide. Numbered embodiment 36 comprises a device of any one of numbered embodiments 1-35, wherein the one or more gases comprises methane and hydrogen sulfide. Numbered embodiment 37 comprises a device of any one of numbered embodiments 1-36, wherein the one or more gases comprises hydrogen, methane, and hydrogen sulfide. Numbered embodiment 38 comprises a device of any one of numbered embodiments 1-37, wherein the device retains at least or about 80% of the carbon dioxide, hydrogen, methane, hydrogen sulfide, or combinations thereof after at least 24 hours. Numbered embodiment 39 comprises a device of any one of numbered embodiments 1-38, wherein the device retains at least or about 80% of the carbon dioxide, hydrogen, methane, hydrogen sulfide, or combinations thereof after at least 48 hours. Numbered embodiment 40 comprises a device of any one of numbered embodiments 1-39, wherein the device retains at least or about 80% of the carbon dioxide, hydrogen, methane, hydrogen sulfide, or combinations thereof after at least 1 week. Numbered embodiment 41 comprises a device of any one of numbered embodiments 1-40, wherein the device retains at least or about 80% of the hydrogen, methane, and hydrogen sulfide after at least 24 hours. Numbered embodiment 42 comprises a device of any one of numbered embodiments 1-41, wherein the device retains at least or about 80% of the hydrogen, methane, and hydrogen sulfide after at least 48 hours. Numbered embodiment 43 comprises a device of any one of numbered embodiments 1-42, wherein the device retains at least or about 80% of the hydrogen, methane, and hydrogen sulfide after at least 1 week. Numbered embodiment 44 comprises a method for assessing a microbiome of an individual, the method comprising: a) collecting a sample from the individual using a device of any one of numbered embodiments 1-43; b) detecting one or more gases in the sample; and c) assessing the microbiome of the individual based on the detection of the one or more gases in the sample. Numbered embodiment 45 comprises a method of any one of numbered embodiments 1-44, wherein the one or more gases comprises carbon dioxide, hydrogen, methane, or hydrogen sulfide. Numbered embodiment 46 comprises a method of any one of numbered embodiments 1-45, wherein a first gas of the one or more gases is hydrogen sulfide. Numbered embodiment 47 comprises a method of any one of numbered embodiments 1-46, wherein the one or more gases comprises carbon dioxide and hydrogen sulfide. Numbered embodiment 48 comprises a method of any one of numbered embodiments 1-47, wherein the one or more gases comprises hydrogen and hydrogen sulfide. Numbered embodiment 49 comprises a method of any one of numbered embodiments 1-48, wherein the one or more gases comprises methane and hydrogen sulfide. Numbered embodiment 50 comprises a method of any one of numbered embodiments 1-49, wherein the one or more gases comprises hydrogen, methane, and hydrogen sulfide. Numbered embodiment 51 comprises a method of any one of numbered embodiments 1-50, further comprising following step c), determining whether an individual has a disease or disorder based on the detection of the one or more gases in the sample. Numbered embodiment 52 comprises a method of any one of numbered embodiments 1-51, wherein the disease or disorder is a metabolic disorder. Numbered embodiment 53 comprises a method of any one of numbered embodiments 1-52, wherein the disease or disorder is diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, glomerular disease, or intestinal methanogenic overgrowth. Numbered embodiment 54 comprises a method of any one of numbered embodiments 1-53, wherein if the one or more gases are elevated by at least about 2-fold, then the individual is determined to have the disease or disorder.

### EXAMPLES

The following examples are included for illustrative purposes only and are not intended to limit the scope of the invention.

### Example 1: Kit for Collecting Gaseous Samples from a Subject

In some embodiments, provided herein is a kit for collecting gaseous samples from a subject. The kit may be mailed or ship to the home or a preferred location of the subject. In some embodiments, the kit includes about 9 collections bags, a mouthpiece, and a consumable solution. In some embodiments, the kit further includes instructions. In some embodiments, the kit includes a machine readable code to be scanned by a device. Scanning of the code may direct a patient to view instructions of how to provide samples for the kit. In some embodiments, the instructions comprise video instructions.

In some embodiments, a subject or patient begins my consuming the entire consumable solution. The consumable solution may be provided in a solid or liquid form. The subject may then be instructed to wait a predetermined amount of time before provided a gaseous sample.

In some embodiments, after consumption of the solution, a subject begins to provide gaseous samples. In some embodiments, a subject connects the provided mouthpiece to the valve of a first collection bag. The subject may then exhale into the mouthpiece to provide a large breath into the collection bag. The collection bag may inflate as the gaseous sample of the breath is transmitted into the collection bag. After the collection bag is inflated, the subject may remove the mouthpiece and set the collection bag into the shipping package.

The subject may then set a timer and wait a predetermined amount of time before attaching the mouthpiece to a second collection bag and repeating the process. In some embodiments, the subject waits 15 minutes before collecting an additional sample. In some embodiments, the patient repeats the process 9 times, providing a total of 9 gaseous samples within 9 collection bags to be analyzed. In some embodiments, between each gaseous sample collection, the subject waits the predetermined amount of time.

In some embodiments, after collection of the samples the subject provides removes the mouthpiece from the collection bag. The subject may then place the collection bags within the shipping materials. In some embodiments, the kit comprises a box which the collection bags are placed into for shipping. The kit may further comprise a shipping label which is preprinted with information regarding where the collection kit will be shipped. The subject may then ship the kit with the collected samples to a testing facility via a preferred carrier. In some embodiments, the one or more mouthpieces and the container to hold the consumable solution are disposed of and not shipped to the testing facility.

### Example 2: Stability Test of Exemplary Embodiment

The collection bag was used to determine stability of gasses retained.

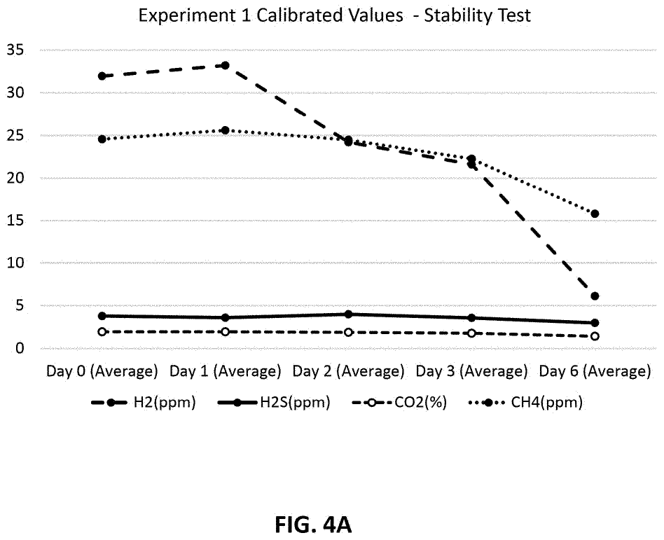

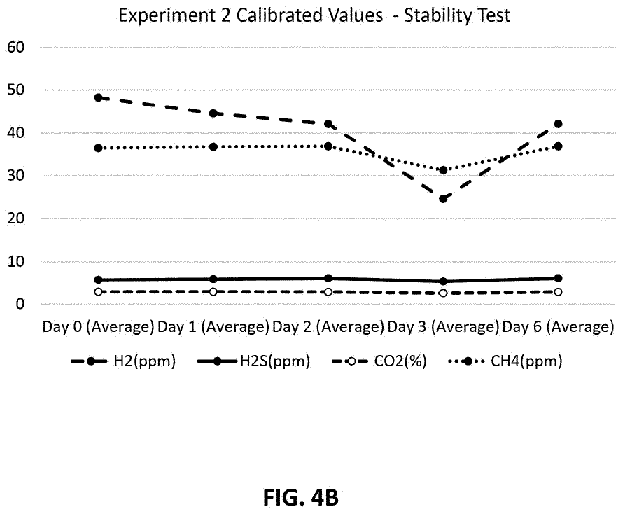

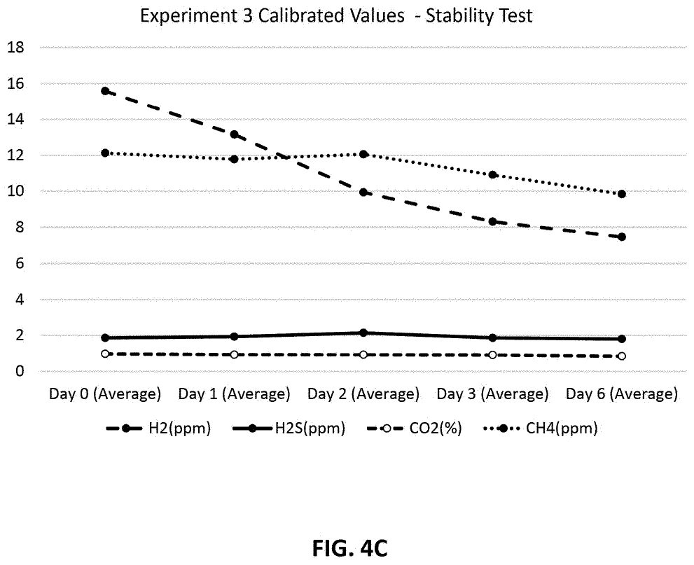

Stability of various concentrations of ambient:gas mixtures were tested including 50:50, 25:75, and 75:25 ambient:gas mixtures. The stability test conducted measured levels of hydrogen (H₂), hydrogen sulfide (H₂S), carbon dioxide (CO₂), and methane (CH₄) retained within a collection bag over a period of 6 days. Data is seen in Tables 1-3 and FIGS. 4A-4C.

TABLE 1
Experiment 1

| | Calibrated Values | | | |
|---|---|---|---|---|
| (50/50) | H₂(ppm) | H₂S(ppm) | CO₂(%) | CH₄(ppm) |
| Day 0 (Average) | 31.94 | 3.80 | 1.96 | 24.56 |
| Day 1 (Average) | 33.20 | 3.60 | 1.96 | 25.59 |
| Day 2 (Average) | 24.21 | 4.00 | 1.89 | 24.48 |
| Day 3 (Average) | 21.62 | 3.58 | 1.79 | 22.27 |
| Day 6 (Average) | 6.13 | 3.00 | 1.43 | 15.80 |

TABLE 2
Experiment 2

| | Calibrated Values | | | |
|---|---|---|---|---|
| (25/75) | H₂(ppm) | H₂S(ppm) | CO₂(%) | CH₄(ppm) |
| Day 0 (Average) | 48.22 | 5.74 | 2.92 | 36.41 |
| Day 1 (Average) | 44.57 | 5.92 | 2.92 | 36.67 |
| Day 2 (Average) | 42.09 | 6.14 | 2.87 | 36.81 |
| Day 3 (Average) | 24.58 | 5.36 | 2.60 | 31.24 |
| Day 6 (Average) | 42.09 | 6.14 | 2.87 | 36.81 |

TABLE 3
Experiment 3

| | Calibrated Values | | | |
|---|---|---|---|---|
| (75/25) | H₂(ppm) | H₂S(ppm) | CO₂(%) | CH₄(ppm) |
| Day 0 (Average) | 15.59 | 1.86 | 0.97 | 12.13 |
| Day 1 (Average) | 13.16 | 1.93 | 0.93 | 11.78 |
| Day 2 (Average) | 9.93 | 2.14 | 0.92 | 12.06 |
| Day 3 (Average) | 8.31 | 1.86 | 0.91 | 10.91 |
| Day 6 (Average) | 7.47 | 1.80 | 0.84 | 9.83 |

The stability test showed that levels of gases retained within the collection bag were sufficient to perform accurate testing and analyzation of the gases held within after a period of at least 2 days. The stability tests show that the collection bag may hold hydrogen sulfide (H₂S) and methane (CH₄) in a stable manner for at least 48 hours. The stability test showed that the collection bag is especially well suited for retaining hydrogen sulfide (H₂S) and methane (CH₄) for periods of time up to 6 days.

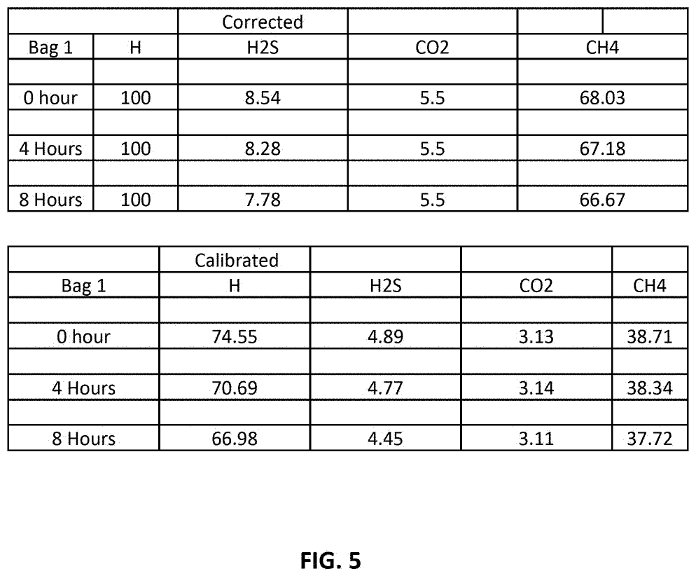

FIG. 5 shows data acquired from a stability test conducted using gaseous samples collected in a preexisting collection bag device. The data was collected in 4 hour increments over a period of 8 hours. The data shows lower levels of retaining hydrogen sulfide (H₂S) and methane (CH₄) in just a matter of hours, making the preexisting bags a poor choice for retaining and transporting gaseous samples. In comparison, the collection bags disclosed herein may retain stable levels of hydrogen sulfide (H₂S) and methane (CH₄) for a period of at least 2 days or more.

### Example 3: Exhaled Hydrogen Sulfide is Increased in Patients with Diarrhea Based on Data from Breath Testing Device

Breath testing for bacterial overgrowth relies on the measurement of hydrogen (H₂) and methane (CH₄). While CH₄ correlates with both presence and severity of constipation, diarrhea does not correlate to H₂. Currently hydrogen sulfide (H₂S), a gas produced by gut bacteria, cannot be measured by conventional instruments. A collection system and instrument described herein was used to determine the relationship between H₂S and diarrhea in patients undergoing breath testing.

Methods

Three groups were compared: 1) healthy controls, 2) subjects with constipation-predominant irritable bowel syndrome defined by Rome IV criteria, and 3) patients with diarrhea predominant symptoms presenting for breath testing. After 12 hours of fasting, subjects provided a baseline breath sample, consumed 10 g lactulose, and gave breath samples every 15 min for 120 min. Breath was collected in a proprietary collection bag rated to retain H₂, CO₂, CH₄, and H₂S and measured using a novel gas chromatography device. H₂S comparisons were made based on cumulative H₂S measurements (area under the curve) and maximum H₂S during the test.

Results

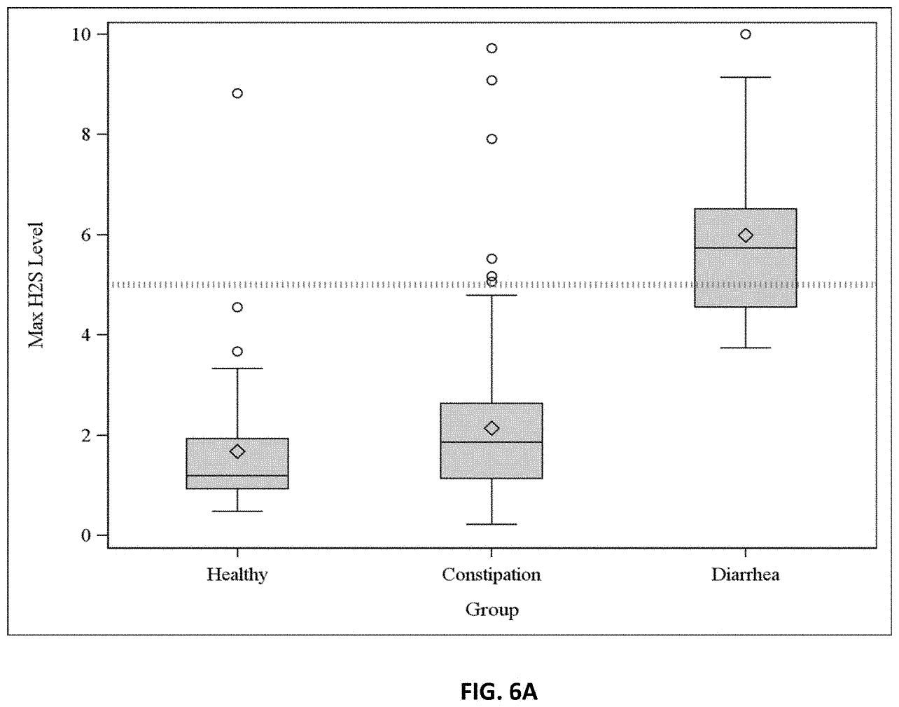

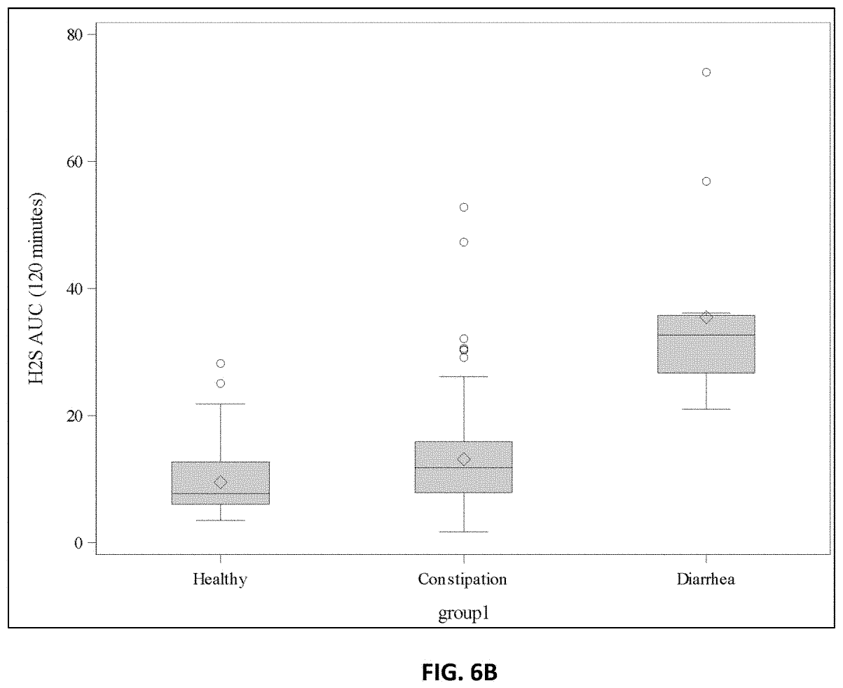

In total, 47 healthy controls (mean age 35.7±11.5 years, 61.7% female), 116 patients with constipation (46.6±12.6, 72.4%) and 12 diarrhea patients (51±17.9, 54.6%) were recruited. Gender was statistically similar between the groups (p=0.24) but healthy controls were younger than other two groups (p<0.001). Mean maximum H₂S level was significantly higher in the diarrhea patients (5.99±1.96 parts per million) as compared to constipated (2.14±1.58) and healthy subjects (1.67±1.38, P<0.001) (FIG. 6A). Similarly, mean H₂S AUC level was significantly higher in the diarrhea patients (35.49±15.34 ppm) as compared to constipated (13.15±8.01) and healthy subjects (9.52±5.21, P<0.001) (FIG. 6B). H₂S≥5 ppm at any point of the test differentiated diarrhea from constipation patients with sensitivity and specificity of 75.0% and 94.8%, respectively. H₂S≥5 ppm at any point of the test differentiated diarrhea from healthy subjects with sensitivity and specificity of 75.0% and 97.9%, respectively.

### CONCLUSION

Using a collection and measurement system described herein, exhaled H₂S levels are significantly higher in patients with diarrhea as compared to healthy controls and patients with constipation. H₂S measurement during clinical breath testing may be a biomarker for diarrhea.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for collecting a microbiome-derived gaseous sample, comprising:
a multilayer composite forming a bag having an interior space for containing the microbiome-derived gaseous sample, the multilayer composite comprising:
an inner layer comprising a first polymer film,
a second layer, adjacent to the inner layer, comprising a foil,
a third layer, adjacent to the second layer, comprising a second polymer film, and
an exterior layer, adjacent to the third layer; and
a valve for in fluid connection with the interior space of the bag, wherein the valve is a one-way valve.

2. The device of claim 1, wherein the first polymer film comprises low-density polyethylene.

3. The device of claim 2, wherein the first polymer film comprises linear low-density polyethylene.

4. (canceled)

5. The device of claim 1, wherein the inner layer and the second layer comprise a metalized film.

6. The device of claim 5, wherein the metalized film comprises biaxially-oriented polyethylene terephthalate.

7. (canceled)

8. The device of claim 1, wherein the third layer and the second layer comprise a metalized film.

9. The device of claim 8, wherein the metalized film comprises biaxially-oriented polyethylene terephthalate.

10. The device of claim 1, wherein the exterior layer comprises nylon.

11. The device of claim 1, wherein the exterior layer comprises a biaxially oriented nylon film (BON).

12. The device of claim 1, further comprising a mouthpiece.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The device of claim 1, wherein the device comprises an oxygen transmission rate of approximately 0.0005 cubic centimeters per 100 square inches per day at 0% relative humidity and 23 degrees Celsius.

29. The device of claim 1, wherein the device comprises a water vapor transmission rate of approximately 0.0005 grams per 100 square inches per day at 90% relative humidity and 40 degrees Celsius.

30. The device of claim 1, wherein the microbiome-derived gaseous sample is a breath sample.

31. (canceled)

32. The device of claim 1, wherein the microbiome-derived gaseous sample comprises carbon dioxide, hydrogen, methane, hydrogen sulfide, or combinations thereof.

33. (canceled)

34. (canceled)

35. (canceled)

36. (canceled)

37. (canceled)

38. The device of claim 32, wherein the device retains at least or about 80% of the carbon dioxide, hydrogen, methane, hydrogen sulfide, or combinations thereof after at least 24 hours.

39. The device of claim 32, wherein the device retains at least or about 80% of the carbon dioxide, hydrogen, methane, hydrogen sulfide, or combinations thereof after at least 48 hours.

40. The device of claim 32, wherein the device retains at least or about 80% of the carbon dioxide, hydrogen, methane, hydrogen sulfide, or combinations thereof after at least 1 week.

41. The device of claim 32, wherein the device retains at least or about 80% of the hydrogen, methane, and hydrogen sulfide after at least 24 hours.

42. The device of claim 32, wherein the device retains at least or about 80% of the hydrogen, methane, and hydrogen sulfide after at least 48 hours.

43. The device of claim 32, wherein the device retains at least or about 80% of the hydrogen, methane, and hydrogen sulfide after at least 1 week.

44-54. (canceled)
